(19) United States
(12) Patent Application Publication
(10) Pub. No.: US 2007/0213332 A1
(43) Pub. Date: Sep. 13, 2007

(54) Prodrugs Of Substituted Amino Heterobicycles Which Modulate The Function Of The Vanilloid-1 Receptor (Vr1)

(76) Inventors: Frank Burkamp, Bishops Stortford (GB); Jose Luis Castro Pineiro, Bishops Stortford (GB); Gregory John Hollingworth, Brentwood (GB)

Correspondence Address: MERCK AND CO., INC, P O BOX 2000, RAHWAY, NJ, 07065-0907, US

(21) Appl. No.: 10/593,766

(22) PCT Filed: Feb. 16, 2005
(86) PCT No.: PCT/GB05/00548

§ 371 Date: Sep. 22, 2006

(30) Foreign Application Priority Data
Feb. 20, 2004 (GB) 0403819.6

(51) Int. Cl. A61K 31/4985; A61P 29/00; C07D 471/04; C07D 487/04

(52) U.S. Cl. 514/242; 514/252.06; 514/299; 544/184; 544/255; 544/282; 548/301.7

(57) ABSTRACT

Compounds of formula (I) which are useful as therapeutic compounds, particularly in the treatment of pain and other conditions ameliorated by the modulation of the function of the vanilloid-1 receptor (VR1).

The present invention is concerned with prodrugs of substituted amino-heterobicycles and pharmaceutically acceptable salts thereof which are useful as therapeutic compounds, particularly in the treatment of pain and other conditions ameliorated by the modulation of the function of the vanilloid-1 receptor (VR1). The prodrugs of this invention have surprising superior physicochemical properties enabling much more drug to be bioavailable.

The pharmacologically active ingredient of chilli peppers has been recognised for some time to be the phenolic amide capsaicin. The application of capsaicin to mucous membranes or when injected intradermally, causes intense burning-like pain in humans. The beneficial effects of topical administration of capsaicin as an analgesic is also well established. However, understanding of the underlying molecular pharmacology mediating these responses to capsaicin has been a more recent development.

The receptor for capsaicin, termed the vanilloid VR1 receptor, was cloned by Caterina and colleagues at UCSF in 1997 (Nature, 398:816, 1997). VR1 receptors are cation channels that are found on sensory nerves that innervate the skin, viscera, peripheral tissues and spinal cord. Activation of VR1 elicits action potentials in sensory fibres that ultimately generate the sensation of pain. Importantly VR1 receptor is activated not only by capsaicin but also by acidic pH and by noxious heat stimuli. It is also sensitized by a number of inflammatory mediators and thus appears to be a polymodal integrator of painful stimuli.

The prototypical VR1 antagonist is capsazepine (Walpole et al., J. Med. Chem., 37:1942, 1994)—VR1 IC₅₀ of 420 nM. A novel series of sub-micromolar antagonists has also been reported recently (Lee et al, Bioorg. Med. Chem., 9:1713, 2001), but these reports provide no evidence for in vivo efficacy. A much higher affinity antagonist has been derived from the ‘ultra-potent’ agonist resiniferatoxin. Iodo-resiniferatoxin (Wahl et al., Mol. Pharmacol., 59:9, 2001) is a nanomolar antagonist of VR1 but does not possess properties suitable for an oral pharmaceutical. This last is also true of the micromolar peptoid antagonists described by Garcia-Martinez (Proc. Natl. Acad. Sci., USA, 99:2374, 2002). Most recently International (PCT) patent publication No. WO 02/08221 has described a novel series of VR1 antagonists, which are stated to show efficacy in a number of animal models. We herein describe another novel series of VR1 modulators. These comprise predominantly VR1 antagonists but encompass VR1 partial antagonists and VR1 partial agonists. Such compounds have been shown to be efficacious in animal models of pain.

The present invention provides compounds of formula (I): 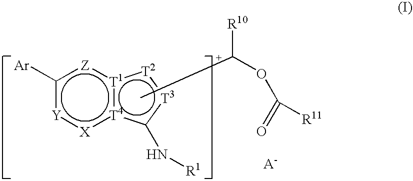
wherein:

one of T¹ and T⁴ is N and the other is C;

one of T² and T³ is N and the other is C(CH₂)ₙR² or N;

X, Y and Z are independently N or C(CH₂)ₙR³;

R¹ is Ar¹ or R¹ is C₁₋₆alkyl optionally substituted with one or two groups Ar¹;

Ar¹ is cyclohexyl, piperidinyl, piperazinyl, morpholinyl, adamantyl, phenyl, naphthyl, a six-membered heteroaromatic ring containing one, two or three nitrogen atoms, a five-membered heteroaromatic ring containing one, two, three or four heteroatoms chosen from O, N and S, at most one O or S atom being present, or a nine- or ten-membered bicyclic heteroaromatic ring in which phenyl or a six-membered heteroaromatic ring as defined above is fused to a six- or five-membered heteroaromatic ring as defined above;

Ar¹ is optionally substituted by one, two or three groups chosen from halogen, hydroxy, cyano, nitro, isonitrile, CF₃, OCF₃, SF₅, C₁₋₆alkyl, C₂₋₆alkenyl, C₂₋₆alkynyl, C₁₋₆alkoxy, C₁₋₆alkylthio, C₁₋₆alkylsulfinyl, C₁₋₆alkylsulfonyl, —NR⁶R⁷, CONR⁶R⁷, —COH, —CO₂H, C₁₋₆alkylcarbonyl, C₁₋₆alkoxycarbonyl, haloC₁₋₆alkyl, haloC₂₋₆alkenyl, haloC₁₋₆alkoxy, hydroxyC₁₋₆ alkyl, aminoC₁₋₆alkyl, cyanoC₁₋₆alkyl, C₃₋₆cycloalkyl, hydroxyC₃₋₆cycloalkyl, aminoC₃₋₆cycloalkyl, haloC₃₋₆cycloalkyl, cyanoC₃₋₆cycloalkyl, haloC₁₋₆alkylcarbonyl, C₁₋₆alkoxycarbonylC₁₋₆alkyl, (halo)(hydroxy)C₁₋₆alkyl, (halo)(hydroxy)C₃₋₆cycloalkyl, phenyl and a five-membered heteroaromatic ring containing one, two or three heteroatoms, at most one O or S atom being present; wherein the phenyl and five-membered heteroaromatic ring are optionally substituted by C₁₋₆alkyl, halo, hydroxy or cyano; when two C₁₋₆alkyl groups substitute adjacent positions on Ar¹ then, together with the carbon atoms to which they are attached, they may form a partially saturated ring containing five or six carbon atoms; when two C₁₋₆alkoxy groups substitute adjacent positions on Ar¹ then, together with the carbon atoms to which they are attached, they may form a partially saturated five- or six-membered ring;

Ar is phenyl, a six-membered heteroaromatic ring containing one, two or three nitrogen atoms or a five-membered heteroaromatic ring containing one, two, three or four heteroatoms chosen from O, N and S, at most one heteroatom being O or S, Ar being optionally substituted by one, two or three groups chosen from halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, haloC₁₋₆alkoxy, hydroxyC₁₋₆alkyl, aminoC₁₋₆alkyl, C₁₋₆alkylcarbonyl and a five-membered heteroaromatic ring containing one, two, three or four heteroatoms chosen from O, N and S, at most one heteroatom being O or S, optionally substituted by C₁₋₆alkyl, halogen, amino, hydroxy or cyano;

R² and R³ are independently hydrogen, halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl, aminoC₁₋₆alkyl, C₁₋₆alkylaminoC₁₋₆alkyl, di(C₁₋₆alkyl)aminoC₁₋₆alkyl, amido, piperidinyl, piperazinyl, C₃₋₆cycloalkyl, morpholinyl, phenyl, a six-membered heteroaromatic ring containing one, two or three nitrogen atoms or a five-membered heteroaromatic ring containing one, two, three or four heteroatoms chosen from O, N and S, at most one O or S atom being present, which phenyl, six-membered heteroaromatic ring and five-membered heteroaromatic ring are optionally substituted by haloC₁₋₆alkyl, C₁₋₆alkyl, hydroxy, halogen, amino or cyano;

R⁶ and R⁷ are independently hydrogen or C₁₋₆alkyl; when both R⁶ and R⁷ are C₁₋₆alkyl then, together with the nitrogen atom to which they are attached, they may form a five or six membered saturated nitrogen containing ring;

n is zero, one, two or three;

R¹⁰ and R¹¹ are independently hydrogen, C₁₋₆alkyl, C₁₋₆alkoxy, C₃₋₆cycloalkyl or NR¹²R¹³;

R¹² and R¹³ are independently hydrogen or C₁₋₆alkyl or R¹² and R¹³, together with the nitrogen atom to which they are attached, may form a nitrogen containing heterocycle; and

A⁻¹ is a pharmaceutically acceptable anion.

In one embodiment Ar¹ is optionally substituted by one, two or three groups chosen from halogen, hydroxy, cyano, nitro, isonitrile, CF₃, OCF₃, SF₅, C₁₋₆alkyl, C₂₋₆alkenyl, C₂₋₆alkynyl, C₁₋₆alkoxy, C₁₋₆alkylthio, C₁₋₆alkylsulfinyl, C₁₋₆alkylsulfonyl, —NR⁶R⁷, CONR⁶R⁷, —COH, —CO₂H, C₁₋₆alkylcarbonyl, C₁₋₆alkoxycarbonyl, haloC₁₋₆alkyl, haloC₂₋₆alkenyl, hydroxyC₁₋₆ alkyl, aminoC₁₋₆alkyl, cyanoC₁₋₆alkyl, C₃₋₆cycloalkyl, hydroxyC₃₋₆cycloalkyl, aminoC₃₋₆cycloalkyl, haloC₃₋₆cycloalkyl, cyanoC₃₋₆cycloalkyl, haloC₁₋₆alkylcarbonyl, C₁₋₆alkoxycarbonylC₁₋₆alkyl, (halo)(hydroxy)C₁₋₆alkyl, (halo)(hydroxy)C₃₋₆cycloalkyl, phenyl and a five-membered heteroaromatic ring containing one, two or three heteroatoms, at most one O or S atom being present; wherein the phenyl and five-membered heteroaromatic ring are optionally substituted by C₁₋₆alkyl, halo, hydroxy or cyano; when two C₁₋₆alkyl groups substitute adjacent positions on Ar¹ then, together with the carbon atoms to which they are attached, they may form a partially saturated ring containing five or six carbon atoms; when two C₁₋₆alkoxy groups substitute adjacent positions on Ar¹ then, together with the carbon atoms to which they are attached, they may form a partially saturated five- or six-membered ring; and

Ar is phenyl, a six-membered heteroaromatic ring containing one, two or three nitrogen atoms or a five-membered heteroaromatic ring containing one, two, three or four heteroatoms chosen from O, N and S, at most one heteroatom being O or S, Ar being optionally substituted by one, two or three groups chosen from halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl, aminoC₁₋₆alkyl, C₁₋₆alkylcarbonyl and a five-membered heteroaromatic ring containing one, two, three or four heteroatoms chosen from O, N and S, at most one heteroatom being O or S, optionally substituted by C₁₋₆alkyl, halogen, amino, hydroxy or cyano.

Preferred core structures are obtained when Y is C(CH₂)ₙR³. In this case it is generally preferred that: X is N, Z is C(CH₂)ₙR³, T⁴ is N, T² and T³ are N or T² is C(CH₂)ₙR² and T³ is N or T² is N and T³ is C(CH₂)ₙR²; or X and Z are C(CH₂)ₙR³ and T², T³ and T⁴ are N; or X is N, Z is C(CH₂)ₙR³, T³ is C(CH₂)ₙR² and T² and T¹ are N; or X, Z, T², T³ and T⁴ are N. Additional core structures include those where X and Z are N, T² and T⁴ are N and T³ is C(CH₂)ₙR²; or X and Z are C(CH₂)ₙR³, T² and T⁴ are N and T³ is C(CH₂)ₙR²; or X is C(CH₂)ₙR³, Z is N, T³ and T⁴ are N and T² is C(CH₂)ₙR².

R¹ is preferably Ar¹ or C₁₋₄alkyl, especially C₁₋₂alkyl, substituted by one or two, preferably one, Ar¹ groups. In particular R¹ can be Ar¹. R¹ may be butyl. R¹ may be cyclohexyl, piperidinyl or adamantyl.

Ar¹ is preferably phenyl, isoquinolyl, piperidinyl, piperazinyl, morpholinyl, cyclohexyl, a six-membered heteroaromatic ring as defined above, such as pyridinyl, or adamantyl, unsubstituted or substituted with one two or three substitutents as defined above. Thus Ar¹ may be phenyl, pyridinyl, piperidinyl, butyl, adamantyl or cyclohexyl. In particular, substitutents are chosen from halogen, hydroxy, cyano, CF₃, SF₅, OCF₃, C₁₋₄alkyl, C₂₋₄alkenyl, C₂₋₄alkynyl, C₁₋₄alkoxy, C₁₋₄alkylthio, C₁₋₄alkylsulfinyl, C₁₋₄alkylsulfonyl, —NR⁶R⁷, cyanoC₁₋₄alkyl, haloC₁₋₄alkylcarbonyl, C₁₋₄alkylcarbonyl, C₁₋₄alkoxycarbonyl, haloC₁₋₄alkyl, haloC₂₋₄alkenyl, hydroxyC₁₋₄alkyl, C₃₋₆cycloalkyl, cyanoC₃₋₆cycloalkyl, (halo)(hydroxy)C₁₋₄alkyl, C₁₋₄alkoxycarbonylC₁₋₄alkyl, phenyl, or a five-membered heteroaromatic ring as defined above where the phenyl or five-membered heteroaromatic ring is unsubstituted or substituted by C₁₋₄alkyl or halogen. More preferably the substitutents are chosen from CF₃, OCF₃, SF₅, halogen, C₁₋₄alkyl, C₁₋₄alkoxy, —NR⁶R⁷, C₁₋₄alkylsulfonyl, cyanoC₁₋₄alkyl, cyanoC₃₋₆cycloalkyl, C₁₋₄alkylpyrazole, halophenyl, haloC₁₋₄alkylcarbonyl, phenyl, C₁₋₄alkoxycarbonylC₁₋₄alkyl, C₃₋₆cycloalkyl, (halo)(hydroxy)C₁₋₄alkyl, hydroxyC₁₋₄alkyl, haloC₁₋₄alkyl and C₁₋₄alkylcarbonyl. Thus the substitutents can be chosen from CF₃, OCF₃, SF₅, methyl, tertiarybutyl, fluorine, chlorine, methoxy, isopropyl, methylthio, hydroxymethyl, methylsulfonyl, acetyl, 1-trifluoromethylethen-1-yl, 2-cyanoprop-2-yl, 1-cyanocycloprop-1-yl, bromine, 2-methylpyrazol-3-yl, 4-fluorophenyl, trifluoromethylcarbonyl, phenyl, 1-ethoxycarbonyl-1-methylethyl, cyclohexyl, 1-hydroxy-1-trifluoromethyl-2,2,2-trifluoroethyl, 1-hydroxy-2-methyl-2-propyl, cyano, ethoxycarbonyl, —OCH₂O—, —CH₂CH₂CH₂— and dimethylamino.

Thus Ar¹ may be phenyl, naphthyl, isoquinolinyl or pyridyl, particularly phenyl or pyridyl, especially phenyl. In particular Ar¹ may be unsubstituted or substituted with one or two substitutents. Ar¹ may be unsubstituted. Ar¹ may be substituted.

Thus preferred R¹ groups include 4-trifluoromethylphenyl, 4-tertiarybutylphenyl, phenyl, 2-trifluoromethylphenyl, 3-chlorophenyl, 3-trifluoromethylphenyl, 2,4-difluorophenyl, 4-methoxyphenyl, 2-isopropylphenyl, 3-methylthiophenyl, 2-naphthyl, 4-trifluoromethoxyphenyl, 1,3-benzodioxol-5-yl, 2-cyanophenyl, 2,4-dichlorophenyl, 3,4-dichlorophenyl, 4-dimethylaminophenyl, 2-methyl-4-chlorophenyl, 3-chloro-4-fluorophenyl, 2-fluoro-6-trifluoromethylphenyl, 2-trifluoromethyl-4-fluorophenyl, 3-trifluoromethyl-4-fluorophenyl, 2-chloro-4-trifluoromethylphenyl, 2,3-dihydro-1H-inden-5-yl, 2,3-dihydro-1,4-benzodioxin-6-yl, 5-isoquinolyl, 2-trifluoromethylpyridin-6-yl and 3-trifluoromethylpyridin-6-yl.

Further preferred R¹ groups include 2-phenylethyl, 3-fluorophenylmethyl, diphenylmethyl, (1S)-1-phenylethyl and 3,4-dichlorophenylmethyl.

Yet further preferred R¹ groups include 4-fluorophenyl, 4-acetylphenyl, 4-methylthiophenyl, 1-trifluoromethylethen-1-ylphenyl, 4-(pentafluorothio)phenyl, 4-chlorophenyl, 4-methylphenyl, 4-hydroxymethylphenyl, 4-methylsulfonylphenyl, 2-chloropyrid-5-yl, 4-(1-cyano-1-methylethyl)phenyl, 4-(1-cyano-1-cyclopropyl)phenyl, 4-bromophenyl, 4-(2-methylpyrazol-3-yl)phenyl, 4-(4-fluorophenyl)phenyl, butyl, adamant-1-yl, 1-trifluoroacetyl-4-piperidinyl, cyclohexyl, 1-phenylpiperidin-4-yl, 4-isopropylphenyl, 4-(1-ethoxycarbonyl-1-methylethyl)phenyl, 4-cyclohexylphenyl, 4-(1-hydroxy-1-trifluoromethyl-2,2,2-trifluoroethyl)phenyl, 4-(1-hydroxy-2-methyl-2-propyl)phenyl, 4-trifluoromethylphenylethyl, 4-cyanophenyl, 4-tert.butylcyclohexyl, 1-ethoxycarbonylpiperidin-4-yl, 3-methylpyridin-6-yl, 2-trifluoromethylpyridin-4-yl, 2-fluoro-4-trifluoromethylphenyl, 4-trifluoromethoxyphenyl and 3-fluoro-4-trifluoromethylphenyl. R¹ can be 4-trifluoromethylphenyl.

Ar is preferably phenyl or a 5- or 6-membered ring containing one or two nitrogen atoms. Ar is more preferably phenyl, pyridyl or imidazolyl, especially pyridyl such as pyrid-2-yl such as 3-substituted pyrid-2-yl. Ar may also be pyridazinyl.

Ar is preferably unsubstituted or substituted with one or two substitutents. More particularly Ar is substituted with one substituent, particularly ortho to the point of attachment to the rest of the molecule.

The substitutents on Ar are preferably chosen from halogen, CF₃, OCF₃, C₁₋₄alkyl, C₁₋₄alkoxy, C₁₋₄alkylcarbonyl, cyano, hydroxyC₁₋₄alkyl and a five-membered heteroaromatic ring as defined above, such as thiazolyl or pyrazolyl, optionally substituted by C₁₋₄alkyl, such as methyl.

The substitutents on Ar are more preferably chosen from halogen, CF₃, OCF₃, C₁₋₄alkyl, C₁₋₄alkoxy, —NR⁶R⁷, haloC₁₋₄alkyl and aminoC₁₋₄alkyl. More preferably they are chosen from halogen, CF₃, C₁₋₂alkoxy and C₁₋₂alkyl, such as CF₃, methyl and methoxy. Thus Ar can be 3-trifluoromethylpyrid-2-yl, 3 methylpyrid-2-yl, 3-methoxypyrid-2-yl, 4-trifluoromethylphenyl or 1-methylimidazol-2-yl. Ar can also be 3-chloropyrid-2-yl, 3-bromopyrid-2-yl, 3-(thiazol-2-yl)pyrid-2-yl, 3-(2-methylpyrazol-3-yl)pyrid-2-yl, 3-acetylpyrid-2-yl, 3-cyanopyrid-2-yl, 3-(2-hydroxyprop-2-yl)pyrid-2-yl, 4-methylpyridazin-3-yl, 4-trifluoromethylpyridazin-3-yl and 2-methoxyphenyl. Ar can be 3-trifluoromethylpyrid-2-yl.

R² is preferably hydrogen, halogen, CF₃, C₁₋₄alkyl, C₁₋₄alkoxy, OCF₃, —NR⁶R⁷, —CO₂H, cyano, amido, phenyl, pyridyl, morpholinyl, imidazolyl or C₁₋₄alkylimidazolyl. These groups may be joined to the rest of the molecule via an ethylene or methylene linker which, when present, is preferably methylene.

R² and R³ are thus preferably hydrogen, halogen, CF₃, C₁₋₂alkyl, C₁₋₂alkoxy, OCF₃ or —NR⁶R⁷. R² and R³ are particularly hydrogen or halogen such as chlorine. R² and R³ are generally hydrogen. Particular embodiments of R² are hydrogen, cyano, bromine, 1-methylimidazol-2-yl, methyl, amido, phenyl, pyrid-4-yl, pyrid-3-yl, morpholin-4-ylmethyl, dimethylaminomethyl, imidazol-1-ylmethyl and carboxyl. R³ may be hydrogen, halogen, such as bromine or chlorine, or cyano.

R⁶ and R⁷ are preferably hydrogen, methyl or ethyl. R⁶ and R⁷ can both be hydrogen, one can be hydrogen and the other can be methyl. In one embodiment they are both methyl.

n is generally 0, 1 or 2, preferably 0 or 1 and most often 0.

In a preferred embodiment T⁴ is N and Y and Z are CR³. More preferably T⁴ is N and Y and Z are CH.

For the avoidance of doubt the moiety CH(R¹⁰)OC(O)R¹¹ is always attached to a nitrogen atom.

A⁻¹ is preferably chloride.

R¹⁰ is preferably hydrogen or C₁₋₄alkyl such as methyl.

R¹¹ is preferably C₁₋₄alkyl, C₁₋₄alkoxy or NR¹²R¹³.

R¹² and R¹³ are preferably hydrogen or C₁₋₄alkyl or form, together with the nitrogen atom to which they are attached, a pyrrolidine ring.

Particular embodiments of R¹¹ are 2-methylprop-2-yl, methyl, prop-2-yl, pyrrolindin-1-yl and 1-methylethoxyl.

Thus the present invention provides a class of compounds of formula I″:

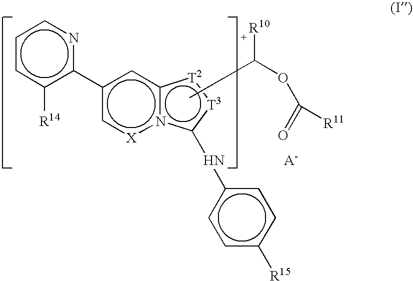

in which X is CH or N;

one of T² and T³ is N and the other is C(CH₂)ₙR²;

R² is hydrogen, halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl, aminoC₁₋₆alkyl, C₁₋₆alkylaminoC₁₋₆alkyl, di(C₁₋₆alkyl)aminoC₁₋₆alkyl, amido, piperidinyl, piperazinyl, C₃₋₆cycloalkyl, morpholinyl, phenyl, a six-membered heteroaromatic ring containing one, two or three nitrogen atoms or a five-membered heteroaromatic ring containing one, two, three or four heteroatoms chosen from O, N and S, at most one O or S atom being present, which phenyl, six-membered heteroaromatic ring and five-membered heteroaromatic ring are optionally substituted by haloC₁₋₆alkyl, C₁₋₆alkyl, hydroxy, halogen, amino or cyano;

R⁶ and R⁷ are independently hydrogen or C₁₋₆alkyl; when both R⁶ and R⁷ are C₁₋₆alkyl then, together with the nitrogen atom to which they are attached, they may form a five or six membered saturated nitrogen containing ring;

n is zero, one, two or three;

R¹⁰ and R¹¹ are independently hydrogen, C₁₋₆alkyl, C₁₋₆alkoxy, C₃₋₆cycloalkyl or NR¹²R¹³ where R¹² and R¹³ are independently hydrogen or C₁₋₆alkyl or R¹² and R¹³, together with the nitrogen atom to which they are attached, form a nitrogen-containing heterocycle;

R¹⁴ and R¹⁵ are independently C₁₋₆alkyl, CF₃, haloC₁₋₆alkyl, halogen, C₁₋₆alkoxy, haloC₁₋₆alkoxy or OCF₃; and

A⁻¹ is a pharmaceutically acceptable anion.

R¹⁰ is preferably hydrogen or C₁₋₄alkyl such as methyl.

R¹¹ is preferably C₁₋₄alkyl, C₁₋₄alkoxy or NR¹²R¹³.

R¹² and R¹³ are preferably hydrogen or C₁₋₄alkyl or form, together with the nitrogen atom to which they are attached, a pyrrolidine ring.

Particular embodiments of R¹¹ are 2-methylprop-2-yl, methyl, prop-2-yl, pyrrolindin-1-yl and 1-methylethoxyl.

R² is preferably hydrogen, C₁₋₆alkyl or cyano. R² may be hydrogen.

R¹⁴ is preferably methyl, CF³ or OCF₃, particularly CF₃.

R¹⁵ is preferably methyl, CF³ or OCF₃, particularly CF₃.

The present invention also provides compounds of formula IA: 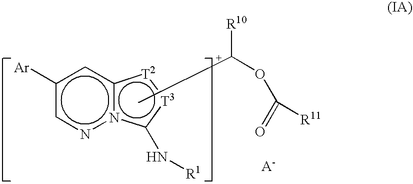
in which T², T³, Ar, R¹, R¹⁰, R¹¹ and A⁻¹ are as defined above. The preferred definitions of these substitutents apply to this subgenus.

Compounds of formula IA are preferred in which R² is hydrogen, Ar is phenyl or pyridyl which is unsubstituted or substituted by methyl, CF₃ or methoxy and R¹ is phenyl substituted generally at the 4-position by CF₃. More particularly Ar is pyridyl, such as pyrid-2-yl, substituted, preferably at the 3-position, by CF₃.

The present invention also provides compounds of formula IB: 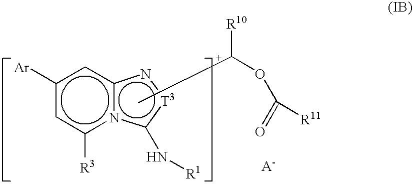
in which Ar, R¹, R³, T³, R¹⁰, R¹¹ and A⁻ are as defined above for formula I including the preferences listed. In one embodiment of the compounds of formula IB, T³ is N.

Compounds of formula IB are preferred in which Ar is pyridyl, particularly when substituted by hydroxy, methyl, methoxy or CF₃, R¹ is phenyl, particularly when substituted by CF₃, and R³ is hydrogen or chlorine. Ar may be substituted by methyl, methoxy or CF₃. Particular preference is for compounds where Ar is pyrid-2-yl substituted at the 3-position and R¹ is 4-trifluoromethylphenyl.

The present invention also provides compounds of formula IC: 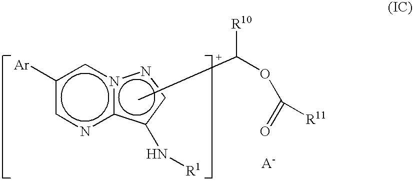
in which Ar, R¹, R¹⁰, R¹¹ and A⁻¹ are as defined above for formula I including the preferences listed. Particularly preferred are compounds in which Ar is pyridyl, particularly when substituted by CF₃, and R¹ is phenyl, particularly when substituted by CF₃. Ar is generally pyrid-2-yl preferably substituted at the 3-position and R¹ is 4-trifluoromethylphenyl.

The present invention also provides compounds of formula ID: 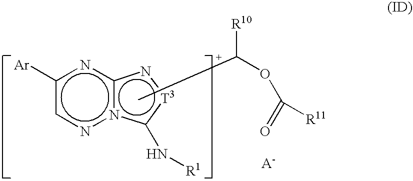
in which Ar, R¹, T³, R¹⁰, R¹¹ and A⁻ are as defined above for formula I including the preferences listed. In one embodiment, T³ in the compounds of formula ID is N. Preferred are compounds in which Ar is pyridyl, particularly when substituted by CF₃ or Cl, and R¹ is phenyl, particularly when substituted by CF₃, cyano or chlorine. Particularly preferred are compounds in which Ar is pyridyl, particularly when substituted by CF₃, and R¹ is phenyl, particularly when substituted by CF₃. Ar is generally pyrid-2-yl preferably substituted at the 3-position and R¹ is 4-trifluoromethylphenyl. R¹ may be 4-chlorophenyl or 4-cyanophenyl.

The present invention provides compounds of formula IE: 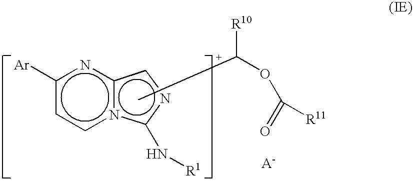
in which Ar, R¹, R¹⁰, R¹¹ and A⁻ are as defined above for formula I including the preferences listed. Particularly preferred are compounds in which Ar is pyridyl, particularly when substituted by CF₃, and R¹ is phenyl, particularly when substituted by CF₃. Ar is generally pyrid-2-yl preferably substituted at the 3-position and R¹ is 4-trifluoromethylphenyl.

Particular embodiments of the invention include:
5-(2,2-dimethylpropanoyloxymethyl)-3-(4-trifluoromethylphenylamino)-7-(3-trifluoromethyl-2-pyridyl)-[1,2,4]triazolo[4,3-b]pyridazinium chloride;
5-(1-(2,2-dimethylpropanoyloxy)-1-ethyl)-3-(4-trifluoromethylphenylamino)-7-(3-trifluoromethyl-2-pyridyl)-[1,2,4]triazolo[4,3-b]pyridazinium chloride;
5-(1-acetoxy-1-ethyl)-3-(4-trifluoromethylphenylamino)-7-(3-trifluoromethyl-2-pyridyl)-[1,2,4]triazolo[4,3-b]pyridazinium chloride;
5-(2-methylpropanoyloxymethyl)-3-(4-trifluoromethylphenylanlino)-7-(3-trifluoromethyl-2-pyridyl)-[1,2,4]triazolo[4,3-b]pyridazinium chloride;
5-(1-pyrrolidinylcarbonyloxymethyl)-3-(4-trifluoromethylphenylamino)-7-(3-trifluoromethyl-2-pyridyl)-[1,2,4]triazolo[4,3-b]pyridazinium chloride;
5-(1-(2-methylpropanoyloxy)-1-ethyl)-3-(4-trifluoromethylphenylamino)-7-(3-trifluoromethyl-2-pyridyl)-[1,2,4]triazolo[4,3-b]pyridazinium chloride;
5-(1-(1-methyl-1-ethoxycarbonyloxy)-1-ethyl)-3-(4-trifluoromethylphenylamino)-7-(3-trifluoromethyl-2-pyridyl)-[1,2,4]triazolo[4,3-b]pyridazinium chloride;
1-{[2,2-dimethylpropanoyloxy]methyl}-3-{4-trifluoromethylphenylamino}-7-[3-trifluoromethylpyridin-2-yl]imidazo[1,2-b]pyridazin-1-ium chloride; and
6-{[2,2-dimethylpropanoyloxy]methyl}-7-{4-trifluoromethylphenylamino}-3-[3-trifluoromethylpyridin-2-yl]imidazo[1,5-b]pyridazin-6-ium chloride.

Examples of compounds which can be converted to prodrugs according to the present invention include:
N-(4-trifluoromethylphenyl)-7-(3-trifluoromethyl-2-pyridyl) [1,2,4]triazolo[4,3-b]pyridazine-3-amine;
N-(4-tert-butyl)phenyl)-7-(3-trifluoromethyl-2-pyridyl)[1,2,4]triazolo[4,3-b]pyridazine-3-amine;
N-phenyl-7-[3-trifluoromethyl-2-pyridinyl][1,2,4]triazolo[4,3-b]pyridazin-3-amine;
N-[2-trifluoromethylphenyl]-7-[3-trifluoromethyl-2-pyridinyl][1,2,4]triazolo[4,3-b]pyridazin-3-amine;
N-(3-chlorophenyl)-7-[3-trifluoromethyl-2-pyridinyl][1,2,4]triazolo[4,3-b]pyridazin-3-amine;
N-[3-trifluoromethylphenyl]-7-[3-trifluoromethyl-2-pyridinyl][1,2,4]triazolo[4,3-b]pyridazin-3-amine;
N-(2,4-difluorophenyl)-7-[3-trifluoromethyl-2-pyridinyl][1,2,4]triazolo[4,3-b]pyridazin-3-amine;
N-[4-methoxyphenyl]-7-[3-trifluoromethyl-2-pyridinyl][1,2,4]triazolo[4,3-b]pyridazin-3-amine;
N-[2-(1-methylethyl)phenyl]-7-[3-trifluoromethyl-2-pyridinyl][1,2,4]triazolo[4,3-b]pyridazin-3-amine;
N-[3-methylsulfanylphenyl]-7-[3-trifluoromethyl-2-pyridinyl][1,2,4]triazolo[4,3-b]pyridazin-3-amine;
N-(2-naphthalenyl)-7-[3-trifluoromethyl-2-pyridinyl][1,2,4]triazolo[4,3-b]pyridazin-3-amine;
N-{4-trifluoromethoxyphenyl}-7-[3-trifluoromethyl-2-pyridinyl][1,2,4]triazolo[4,3-b]pyridazin-3-amine;
N-(2-phenylethyl)-7-[3-trifluoromethyl-pyridinyl][1,2,4]triazolo[4,3-b]pyridazin-3-amine;
N-(1,3-benzodioxol-5-yl)-7-[3-trifluoromethyl-2-pyridinyl][1,2,4]triazolo[4,3-b]pyridazin-3-amine;
N-[3-fluorophenylmethyl]-7-[3-trifluoromethyl-2-pyridinyl][1,2,4]triazolo[4,3-b]pyridazin-3-amine;
2-({7-[3-trifluoromethyl-2-pyridinyl][1,2,4]triazolo[4,3-b]pyridazin-3-yl}amino)benzonitrile;
N-(diphenylmethyl)-7-[3-trifluoromethyl-2-pyridinyl][1,2,4]triazolo[4,3-b]pyridazin-3-amine;
N-[(1S)-1-phenylethyl]-7-[3-trifluoromethyl-2-pyridinyl][1,2,4]triazolo[4,3-b]pyridazin-3-amine;
N-(2,4-dichlorophenyl)-7-[3-trifluoromethyl-2-pyridinyl][1,2,4]triazolo[4,3-b]pyridazin-3-amine;
N-(3,4-dichlorophenyl)-7-[3-trifluoromethyl-2-pyridinyl][1,2,4]triazolo[4,3-b]pyridazin-3-amine;
N-[4-dimethylaminophenyl]-N-{7-[3-trifluoromethyl-2-pyridinyl][1,2,4]triazolo[4,3-b]pyridazin-3-yl}amine;
N-[(3,4-dichlorophenyl)methyl]-7-[3-trifluoromethyl-2-pyridinyl][1,2,4]triazolo[4,3-b]pyridazin-3-amine;
N-(4-chloro-2-methylphenyl)-7-[3-trifluoromethyl-2-pyridinyl][1,2,4]triazolo[4,3-b]pyridazin-3-amine;
N-(3-chloro-4-fluorophenyl)-7-[3-trifluoromethyl-2-pyridinyl][1,2,4]triazolo[4,3-b]pyridazin-3-amine;
N-[2-fluoro-6-trifluoromethylphenyl]-7-[3-trifluoromethyl-2-pyridinyl][1,2,4]triazolo[4,3-b]pyridazin-3-amine;
N-[4-fluoro-2-trifluoromethylphenyl]-7-[3-trifluoromethyl-2-pyridinyl][1,2,4]triazolo[4,3-b]pyridazin-3-amine;
N-[4-fluoro-3-trifluoromethylphenyl]-7-[3-trifluoromethyl-2-pyridinyl][1,2,4]triazolo[4,3-b]pyridazin-3-amine;
N-[2-chloro-4-trifluoromethylphenyl]-7-[3-trifluoromethyl-2-pyridinyl][1,2,4]triazolo[4,3-b]pyridazin-3-amine;
N-(2,3-dihydro-1H-inden-5-yl)-7-[3-trifluoromethyl-2-pyridinyl][1,2,4]triazolo[4,3-b]pyridazin-3-amine;
N-(2,3-dihydro-1,4-benzodioxin-6-yl)-7-[3-trifluoromethyl-2-pyridinyl][1,2,4]triazolo[4,3-b]pyridazin-3-amine;
N-(4-trifluoromethylphenyl)-7-(3-methyl-2-pyridyl)[1,2,4]triazolo[4,3-b]pyridazine-3-amine;
5-chloro-7-(3-methyl-2-pyridyl)-N-(4-trifluoromethylphenyl)[1,2,4]triazolo[4,3-a]pyridin-3-amine;
5-chloro-7-(2-methoxyphenyl)-N-(4-trifluoromethylphenyl) [1,2,4]triazolo[4,3-a]pyridin-3-amine;
5-chloro-N-(4-trifluoromethylphenyl)-7-(3-trifluoromethyl-2-pyridyl)[1,2,4]triazolo[4,3-a]pyridin-3-amine;
6-chloro-N-(5-isoquinolyl)-7-(4-trifluoromethylphenyl)[1,2,4]triazolo[4,3-b]pyridazin-3-amine;
7-(3-methyl-2-pyridyl)-N-(4-trifluoromethylphenyl)[1,2,4]triazolo[4,3-a]pyridin-3-amine;
7-(3-trifluoromethyl-2-pyridyl)-N-(4-trifluoromethylphenyl)[1,2,4]triazolo[4,3-a]pyridin-3-amine;
7-(2-methoxyphenyl)-N-(4-trifluoromethylphenyl)[1,2,4]triazolo[4,3-a]pyridin-3-amine;
N-(5-isoquinolyl)-7-(4-trifluoromethylphenyl)[1,2,4]triazolo[4,3-b]pyridazine-3-amine;
7-(3-trifluoromethyl-2-pyridyl)-N-(5-trifluoromethyl-2-pyridyl)[1,2,4]triazolo[4,3-b]pyridazine-3-amine;
N-(4-trifluoromethylphenyl)-6-(3-trifluoromethylpyrid-2-yl)pyrazolo[1,5-a]pyrimidin-3-amine;
4-trifluoromethylphenyl-3-(3-trifluoromethylpyridin-2-yl)imidazo[1,5-b]pyridazin-7-ylamine;
N-[4-trifluoromethylphenyl]-7-[3-trifluoromethylpyridin-2-yl]imidazo[1,2-b]pyridazin-3-amine;
7-[3-trifluoromethylpyridin-2-yl]-N-[5-trifluoromethylpyridin-2-yl]imidazo[1,2-b]pyridazin-3-amine;
N-(5-methylpyridin-2-yl)-7-[3-trifluoromethylpyridin-2-yl]imidazo[1,2-b]pyridazin-3-amine;
[7-(3-methylpyridin-2-yl)-[1,2,4]triazolo[4,3-b][1,2,4]triazin-3-yl]-(4-trifluoromethyl-phenyl)amine; and
[7-(1-methyl-1H-imidazol-2-yl)[1,2,4]triazolo[4,3-b]pyridazin-3-yl]-(4-trifluoromethylphenyl)amine.

Further preferred compounds which can be converted to pro drugs of the invention include:
7-(3-chloro-2-pyridyl)-N-(4-trifluoromethylphenyl)[1,2,4]triazolo[4,3-b]pyridazine-3-amine;
7-(3-bromo-2-pyridyl)-N-(4-trifluoromethylphenyl) [1,2,4]triazolo[4,3-b]pyridazine-3-amine;
7-[3-(1,3-thiazol-2-yl)pyridin-2-yl]-N-(4-trifluoromethylphenyl)[[1,2,4]triazolo[4,3-b]pyridazine-3-amine;
7-[3-(1-methyl-1H-pyrazol-5-yl)pyridin-2-yl)-N-(4-trifluoromethylphenyl) [1,2,4]triazolo[4,3-b]pyridazine-3-amine;
7-(3-ethoxycarbonyl-2-pyridyl)-N-(4-trifluoromethylphenyl)[1,2,4]triazolo[4,3-b]pyridazine-3-amine;
7-(3-cyano-2-pyridyl)-N-(4-trifluoromethylphenyl)[1,2,4]triazolo[4,3-b]pyridazine-3-amine;
N-(4-chlorophenyl)-7-(3-trifluoromethyl-2-pyridyl)[1,2,4]triazolo[4,3-b]pyridazine-3-amine;
N-(4-tolyl)-7-(3-trifluoromethyl-2-pyridyl)[1,2,4]triazolo[4,3-b]pyridazine-3-amine;
N-(4-(2-hydroxyethyl)phenyl)-7-(3-trifluoromethyl-2-pyridyl)[1,2,4]triazolo[4,3-b]pyridazine-3-amine;
N-(4-methylsulfonylphenyl)-7-(3-trifluoromethyl-2-pyridyl)[1,2,4]triazolo[4,3-b]pyridazine-3-amine;
N-(2-chloro-5-pyridyl)-7-(3-trifluoromethyl-2-pyridyl)[1,2,4]triazolo[4,3-b]pyridazine-3-amine;
N-(4-(1-cyano-1-methylethyl)phenyl)-7-(3-trifluoromethyl-2-pyridyl)[1,2,4]triazolo[4,3-b]pyridazine-3-amine;
N-(4-(1-cyclopropylphenyl)-7-(3-trifluoromethyl-2-pyridyl)[1,2,4]triazolo[4,3-b]pyridazine-3-amine;
N-(4-bromophenyl)-7-(3-trifluoromethyl-2-pyridyl)[1,2,4]triazolo[4,3-b]pyridazine-3-amine;
N-(4-(2-methyl-3-pyrazolo)phenyl)-7-(3-trifluoromethyl-2-pyridyl) [1,2,4]triazolo[4,3-b]pyridazine-3-amine;
N-(4-(4-fluorophenyl)phenyl)-7-(3-trifluoromethyl-2-pyridyl)[1,2,4]triazolo[4,3-b]pyridazine-3-amine;
N-butyl-7-(3-trifluoromethyl-2-pyridyl)[1,2,4]triazolo[4,3-b]pyridazine-3-amine;
N-(1-adamantyl)-7-(3-trifluoromethyl-2-pyridyl)[1,2,4]triazolo[4,3-b]pyridazine-3-amine;
N-(1-trifluoroacetyl-4-piperidinyl)-7-(3-trifluoromethyl-2-pyridyl)[1,2,4]triazolo[4,3-b]pyridazine-3-amine;
N-(1-cyclohexyl)-7-(3-trifluoromethyl-2-pyridyl)[1,2,4]triazolo[4,3-b]pyridazine-3-amine;
N-(1-phenyl-4-piperidinyl)-7-(3-trifluoromethyl-2-pyridyl)[1,2,4]triazolo[4,3-b]pyridazine-3-amine;
N-(4-trifluoromethylphenyl)-7-(2-cyanophenyl)-[1,2,4]triazolo[4,3-b]pyridazine-3-amine;
N-(4-trifluoromethylphenyl)-7-(3-(1-hydroxy-1-methylethyl)-2-pyridyl-[1,2,4]triazolo[4,3-b]pyridazine-3-amine;
N-(4-(1-methylethyl)phenyl)-7-(3-trifluoromethyl-2-pyridyl)[1,2,4]triazolo[4,3-b]pyridazine-3-amine;
N-(4-(1-ethoxycarbonyl-1-methylethyl)phenyl)-7-(3-trifluoromethyl-2-pyridyl) [1,2,4]triazolo[4,3-b]pyridazine-3-amine;
N-(4-cyclohexylphenyl)-7-(3-trifluoromethyl-2-pyridyl)[1,2,4]triazolo[4,3-b]pyridazine-3-amine;
N-(4-(1-hydroxy-1-trifluoromethyl-2,2,2-trifluoroethyl)phenyl)-7-(3-trifluoromethyl-2-pyridyl)[1,2,4]triazolo[4,3-b]pyridazine-3-amine;
N-(4-(1-hydroxy-2-methyl-2-propyl)phenyl)-7-(3-trifluoromethyl-2-pyridyl) [1,2,4]triazolo[4,3-b]pyridazine-3-amine;
N-(2-4-trifluoromethylphenylethyl)-7-(3-trifluoromethyl-2-pyridyl)[1,2,4]triazolo[4,3-b]pyridazine-3-amine;
N-(trans)-(4-tert.-butylcyclohexyl)-7-(3-trifluoromethyl-2-pyridyl)[1,2,4]triazolo[4,3-b]pyridazine-3-amine;
N-(1-ethoxycarbonyl-4-piperidinyl)-7-(3-trifluoromethyl-2-pyridyl) [1,2,4]triazolo[4,3-b]pyridazine-3-amine;
7-(4-methylpyridazin-3-yl)-N-[4-trifluoromethylphenyl][1,2,4]triazolo[4,3-b]pyridazin-3-amine;
N-[4-trifluoromethylphenyl]-7-[5-trifluoromethylpyrimidin-4-yl][1,2,4]triazolo[4,3-b]pyridazin-3-amine;
5-bromo-N-[4-trifluoromethylphenyl]-3-[3-trifluoromethylpyridin-2-yl]imidazo[1,5-b]pyridazin-7-amine;
5-(1-methyl-1H-imidazol-2-yl)-N-[4-trifluoromethylphenyl]-3-[3-trifluoromethyl pyridin-2-yl]imidazo[1,5-b]pyridazin-7-amine;
N-(4-chlorophenyl)-3-[3-trifluoromethylpyridin-2-yl]imidazo[1,5-b]pyridazin-7-amine;
5-methyl-N-[4-trifluoromethylphenyl]-3-[3-trifluoromethylpyridin-2-yl]imidazo[1,5-b]pyridazin-7-amine;
7-{[4-trifluoromethylphenyl]amino}-3-[3-trifluoromethylpyridin-2-yl]imidazo[1,5-b]pyridazine-5-carbonitrile;
7-{[4-trifluoromethylphenyl]amino}-3-[3-trifluoromethylpyridin-2-yl]imidazo[1,5-b]pyridazine-5-carboxamide;
3-(3-methylpyridin-2-yl)-N-[4-trifluoromethylphenyl]imidazo[1,5-b]pyridazin-7-amine;
3-(3-methylpyridin-2-yl)-7-{[4-trifluoromethylphenyl]amino}imidazo[1,5-b]pyridazine-5-carbonitrile;
5-phenyl-N-[4-trifluoromethylphenyl]-3-[3-trifluoromethylpyridin-2-yl]imidazo[1,5-b]pyridazin-7-amine;
5-pyridin-4-yl-N-[4-trifluoromethylphenyl]-3-[3-trifluoromethylpyridin-2-yl]imidazo[1,5-b]pyridazin-7-amine;
5-pyridin-3-yl-N-[4-trifluoromethylphenyl]-3-[3-trifluoromethylpyridin-2-yl]imidazo[1,5-b]pyridazin-7-amine
5-(morpholin-4-ylmethyl)-N-[4-trifluoromethylphenyl]-3-[3-trifluoromethylpyridin-2-yl]imidazo[1,5-b]pyridazin-7-amine;
5-[dimethylaminomethyl]-N-[4-trifluoromethylphenyl]-3-[3-trifluoromethylpyridin-2-yl]imidazo[1,5-b]pyridazin-7-amine;
5-(1H-imidazol-1-ylmethyl)-N-[4-trifluoromethylphenyl]-3-[3-trifluoromethyl pyridin-2-yl]imidazo[1,5-b]pyridazin-7-amine;
7-{[4-trifluoromethylphenyl]amino}-3-[3-trifluoromethylpyridin-2-yl]imidazo[1,5-b]pyridazine-5-carboxylic acid;
7-[1-oxido-3-trifluoromethylpyridin-2-yl]-N-[4-trifluoromethylphenyl]-imidazo [1,2-b]pyridazin-3-amine;
2-bromo-N-[4-trifluoromethylphenyl]-7-[3-trifluoromethylpyridin-2-yl]imidazo[1,2-b]pyridazin-3-amine;
3-{[4-trifluoromethylphenyl]amino}-7-[3-trifluoromethylpyridin-2-yl]imidazo[1,2-b]pyridazine-2-carbonitrile;
2-methyl-N-[4-trifluoromethylphenyl]-7-[3-trifluoromethylpyridin-2-yl]imidazo[1,2-b]pyridazin-3-amine;
7-[3-trifluoromethylpyridin-2-yl]-N-[6-trifluoromethylpyridin-3-yl]imidazo[1,2-b]pyridazin-3-amine;
N-(4-chlorophenyl)-7-[3-trifluoromethylpyridin-2-yl]imidazo[1,2-b]pyridazin-3-amine;
N-[2-fluoro-4-trifluoromethylphenyl]-7-[3-trifluoromethylpyridin-2-yl]imidazo[1,2-b]pyridazin-3-amine;
N-(6-methylpyridin-3-yl)-7-[3-trifluoromethylpyridin-2-yl]imidazo[1,2-b]pyridazin-3-amine;
N-[4-trifluoromethoxyphenyl]-7-[3-trifluoromethylpyridin-2-yl]imidazo[1,2-b]pyridazin-3-amine;
N-[3-fluoro-4-trifluoromethylphenyl]-7-[3-trifluoromethylpyridin-2-yl]imidazo[1,2-b]pyridazin-3-amine;
7-(3-chloropyridin-2-yl)-N-[4-trifluoromethylphenyl]imidazo[1,2-b]pyridazin-3-amine;
N-(4-chlorophenyl)-7-(3-chloropyridin-2-yl)imidazo[1,2-b]pyridazin-3-amine;
[7-(3-trifluoromethyl-pyridin-2-yl)-[1,2,4]triazolo[4,3-b][1,2,4]triazin-3-yl]-(4-trifluoromethyl-phenyl)-amine;
N-(4-chlorophenyl)-7-[3-trifluoromethylpyridin-2-yl][1,2,4]triazolo[4,3-b][1,2,4]triazin-3-amine;
4-({7-[3-trifluoromethylpyridin-2-yl][1,2,4]triazolo[4,3-b][1,2,4]triazin-3-yl}amino)benzonitrile;
7-(3-chloropyridin-2-yl)-N-[4-trifluoromethylphenyl][1,2,4]triazolo[4,3-b][1,2,4]triazin-3-amine;
N-(4-chlorophenyl)-7-(3-chloropyridin-2-yl) [1,2,4]triazolo[4,3-b][1,2,4]triazin-3-amine;
3-(3-methylpyridin-2-yl)-N-[4-trifluoromethylphenyl]imidazo[1,2-b][1,2,4]triazin-7-amine;
3-(3-chloropyridin-2-yl)-N-[4-trifluoromethylphenyl]imidazo[1,2-b][1,2,4]triazin-7-amine;
N-[4-trifluoromethylphenyl]-3-[3-trifluoromethylpyridin-2-yl]imidazo[1,2-b][1,2,4]triazin-7-amine;
N-[4-trifluoromethylphenyl]-7-[3-trifluoromethylpyridin-2-yl]imidazo[1,2a]pyridin-3-amine;
N-[4-trifluoromethylphenyl]-2-[3-trifluoromethylpyridin-2-yl]imidazo[1,5-a]pyrimidin-6-amine; and
N-(4-trifluoromethylphenyl)-7-(2-methoxyphenyl)[1,2,4]triazolo[4,3-b]pyridazine-3-amine.

Further compounds which can be converted to prodrugs of the invention include:
N-(4-fluorophenyl)-7-(3-trifluoromethylpyrid-2-yl)[1,2,4]triazolo[4,3-b]pyridazine-3-amine;
N-(4-acetylphenyl-7-(3-trifluoromethylpyrid-2-yl)[1,2,4]triazolo[4,3-b]pyridazine-3-amine;
N-(3-trifluoromethylpyrid-4-yl)-7-(3-trifluoromethylpyrid-2-yl)[1,2,4]triazolo[4,3-b]pyridazine-3-amine;
N-(4-methylthiophenyl)-7-(3-trifluoromethylpyrid-2-yl)[1,2,4]triazolo[4,3-b]pyridazine-3-amine;
N-(1-trifluoromethylethen-1-yl)-7-(3-trifluoromethylpyrid-2-yl)[1,2,4]triazolo[4,3-b]pyridazine-3-amine;
N-(3-trifluoromethylpyrid-6-yl)-7-(3-trifluoromethylpyrid-2-yl)[1,2,4]triazolo[4,3-a]pyridine-3-amine;
N-(4-pentafluorothiophenyl)-7-(3-trifluoromethylpyrid-2-yl) [1,2,4]triazolo[4,3-b]pyridazine-3-amine; and
N-(4-cyanophenyl)-7-(3-trifluoromethylpyrid-2-yl)[1,2,4]triazolo[4,3-b]pyridazine-3-amine.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy. “Alkylthio”, “alkylsulfinyl” and “alkylsulfonyl” shall be construed in an analogous manner.

As used herein, the term “hydroxyC₁₋₆alkyl” means a C₁₋₆alkyl group in which one or more (in particular 1 to 3, and especially 1) hydrogen atoms have been replaced by hydroxy groups. Particularly preferred are hydroxyC₁₋₃alkyl groups, for example, CH₂OH, CH₂CH₂OH, CH(CH₃)OH or C(CH₃)₂OH, and most especially CH₂OH. “Aminoalkyl”, “cyanoalkyl” and “(halo)(hydroxy)alkyl” shall be construed in an analogous manner.

As used herein, the terms “haloC₁₋₆alkyl” and “haloC₁₋₆alkoxy” means a C₁₋₆alkyl or C₁₋₆alkoxy group in which one or more (in particular, 1 to 3) hydrogen atoms have been replaced by halogen atoms, especially fluorine or chlorine atoms. Preferred are fluoroC₁₋₆alkyl and fluoroC₁₋₆alkoxy groups, in particular, fluoroC₁₋₃alkyl and fluoroC₁₋₃alkoxy groups, for example, CF₃, CH₂CH₂F, CH₂CHF₂, CH₂CF₃, OCF₃, OCH₂CH₂F, OCH₂CHF₂ or OCH₂CF₃, and most especially CF₃, OCF₃ and OCH₂CF₃.

As used herein, the terms “alkenyl” and “alkynyl” as a group or part of a group means that the group is straight or branched. Examples of suitable alkenyl groups include vinyl and allyl. A suitable alkynyl group is acetylene or propargyl.

As used herein, the term “cycloalkyl” as a group or part of a group means that the group contains a cyclic portion. Examples of suitable cycloalkyl groups include cyclopropyl, methylcyclopropyl, cyclobutyl, cyclopentyl and cyclohexyl. Cyclohexyl groups, when substituted, may have a cis or trans configuration. Terms such as “halocycloalkyl”, “cyanocycloalkyl”, “hydroxycycloalkyl”, “aminocycloalkyl” and “(halo)(hydroxy)cycloalkyl” shall be construed analogously to the above definitions for alkyl derivatives.

When used herein, the term “halogen” means fluorine, chlorine, bromine and iodine. The most preferred halogens are fluorine and chlorine.

When used herein, the term “carboxy” as a group or part of a group denotes CO₂H.

When used herein, the term “C₁₋₆alkoxycarbonyl” denotes a C₁₋₆alkoxy or a haloC₁₋₆alkoxy radical attached via the oxygen atom thereof to a carbonyl (C═O) radical thus forming a C₁₋₆alkoxycarbonyl or haloC₁₋₆alkoxycarbonyl radical. Suitable examples of such esterified carboxy groups include, for example, methoxycarbonyl, ethoxycarbonyl, propoxycarbonyl, isopropoxycarbonyl and tert-butoxycarbonyl.

Examples of 6-membered heterocycles are pyridine, pyrimidine, pyrazine, pyridazine and triazine.

Examples of 5-membered heterocycles are thiophene, furan, pyrrole, imidazole, pyrazole, oxazole, isoxazole, thiazole, isothiazole, 1,2,3-triazole, 1,2,4-triazole, oxadiazole, thiadiazole and tetrazole.

As used herein, the term “fused 9 or 10 membered bicyclic heteroaromatic ring system” means a 5,6-, 6,5- or 6,6-fused ring system wherein one or both rings contain ring heteroatoms. The ring system is preferably aromatic or partially saturated, thus the ring system preferably comprises an aromatic 6-membered ring fused to a 5- or 6-membered ring which may be unsaturated, partially saturated or saturated. When the ring system contains more than one ring heteroatom at least one such heteroatom is nitrogen. It will be appreciated that where one of the ring heteroatoms is a nitrogen atom, such heteroatom may be at the bridgehead position of the fused ring system. It will also be appreciated that where one of the ring heteroatoms in a saturated ring is sulfur, such heteroatom may be oxidized to a S(O) or S(O)₂ moiety. Likewise, any carbon atom in a saturated ring may be oxidized to a C═O moiety.

Suitable examples of a “fused 9 or 10 membered heterobicyclic ring system” include isoquinolinyl, quinolinyl, cinnolinyl, phthalazinyl, quinazolinyl, quinoxalinyl, naphthyridinyl, indolyl, isoindolyl, benzofuranyl, benzothiophenyl, benzimidazolyl, indazolyl, benzoxazolyl, benzisoxazolyl, benzothiazolyl, benzisothiazolyl, benzotriazole, pyridopyridazinyl, pyridopyrimidinyl, pyridopyrazinyl, pyrrolopyridinyl, furopyridinyl, thienopyridinyl, pyrrolopyridazinyl, furopyridazinyl, thienopyridazinyl, pyrrolopyrimidinyl, furopyrimidinyl, thienopyrimidinyl, pyrrolopyrazinyl, furopyrazinyl, thienopyrazinyl, imidazopyridinyl, pyrazolopyridinyl, oxazolopyridinyl, isoxazolopyridinyl, thiazolopyridinyl, isothiazolopyridinyl, imidazopyridazinyl, pyrazolopyridazinyl, oxazolopyridazinyl, isoxazolopyridazinyl, thiazolopyridazinyl, isothiazolopyridazinyl, imidazopyrimidinyl, pyrazolopyrimidinyl, oxazolopyrimidinyl, isoxazolopyrimidinyl, thiazolopyrimidinyl, isothiazolopyrimidinyl, imidazopyrazinlyl, pyrazolopyrazinyl, oxazolopyrazinyl, isoxazolopyrazinyl, thiazolopyrazinyl, isothiazolopyrazinyl, triazolopyridinyl, benzotriazolyl, quinolinonyl, isoquinolinonyl, dihydroquinolinonyl, dihydroisoquinolinonyl, tetrahydroquinolinyl, tetrahydroisoquinolinyl, dihydroquinazolinonyl, dihydrobenzoxainonyl, dihydrobenzothiadiazine oxide and dihydrobenzothiadiazine dioxide.

The pharmaceutically acceptable anion may be derived from hydrochloric acid, fumaric acid, p-toluenesulphonic acid, maleic acid, succinic acid, acetic acid, citric acid, tartaric acid, carbonic acid, phosphoric acid or sulphuric acid.

The present invention also includes within its scope N-oxides of the compounds of formula (I) above. In general, such N-oxides may be formed on any available nitrogen atom. The N-oxides may be formed by conventional means, such as reacting the compound of formula (J) with oxone in the presence of wet alumina.

The present invention includes within its scope solvates of the compounds of formula (I).

The compounds according to the invention may have one or more asymmetric centres, and may accordingly exist both as enantiomers and as diastereoisomers. It is to be understood that all such isomers and mixtures thereof are encompassed within the scope of the present invention. Furthermore, the compounds of formula (I) may also exist in tautomeric forms and the invention includes within its scope both mixtures and separate individual tautomers.

The present invention further provides pharmaceutical compositions comprising one or more compounds of formula (I) in association with a pharmaceutically acceptable carrier or excipient.

Preferably the compositions according to the invention are in unit dosage forms such as tablets, pills, capsules, powders, granules, sterile parenteral solutions or suspensions, metered aerosol or liquid sprays, drops, ampoules, auto-injector devices, suppositories, creams or gels; for oral, parenteral, intrathecal, intranasal, sublingual, rectal or topical administration, or for administration by inhalation or insufflation. Oral compositions such as tablets, pills, capsules or wafers are particularly preferred. For preparing solid compositions such as tablets, the principal active ingredient is mixed with a pharmaceutical carrier, e.g. conventional tabletting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation composition is then subdivided into unit dosage forms of the type described above containing from 0.1 to about 500 mg of the active ingredient of the present invention. Favoured unit dosage forms contain from 1 to 500 mg, for example 1, 5, 10, 25, 50, 100, 300 or 500 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of painful conditions such as those listed below, a suitable dosage level is about 1.0 mg to 15 g per day, preferably about 5.0 mg to 1 g per day, more preferably about 5 mg to 500 mg per day, especially 10 mg to 100 mg per day. The compounds may be administered on a regimen of 1 to 4 times per day.

It will be appreciated that the amount of a compound of formula (I) required for use in any treatment will vary not only with the particular compounds or composition selected but also with the route of administration, the nature of the condition being treated, and the age and condition of the patient, and will ultimately be at the discretion of the attendant physician.

The invention further provides a compound of formula (I) as defined above for use in treatment of the human or animal body. Preferably, said treatment is for a condition which is susceptible to treatment by modulation (preferably antagonism) of VR1 receptors.

The compounds of the present invention will be of use in the prevention or treatment of diseases and conditions in which pain and/or inflammation predominates, including chronic and acute pain conditions. Such conditions include rheumatoid arthritis; osteoarthritis; post-surgical pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labour pain, chronic pelvic pain, chronic prostatitis and endometriosis; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; itching conditions including piuritis, itch due to hemodialysis, and contact dermatitis; pain (as well as broncho-constriction and inflammation) due to exposure (e.g. via ingestion, inhalation, or eye contact) of mucous membranes to capsaicin and related irritants such as tear gas, hot peppers or pepper spray; neuropathic pain conditions such as diabetic neuropathy, chemotherapy-induced neuropathy and post-herpetic neuralgia; “non-painful” neuropathies; complex regional pain syndromes; pain associated with carcinoma, often referred to as cancer pain; central nervous system pain, such as pain due to spinal cord or brain stem damage, low back pain, sciatica and ankylosing spondylitis; gout; scar pain; irritable bowel syndrome; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis, asthma and rhinitis, including allergic rhinitis such as seasonal and perennial rhinitis, and non-allergic rhinitis; autoimmune diseases; and immunodeficiency disorders.

Thus, according to a further aspect, the present invention provides a compound of formula (I) for use in the manufacture of a medicament for the treatment or prevention of physiological disorders that may be ameliorated by modulating VR1 activity.

The present invention also provides a method for the treatment or prevention of physiological disorders that may be ameliorated by modulating VR1 activity, which method comprises administration to a patient in need thereof of an effective amount of a compound of formula (I) or a composition comprising a compound of formula (I).

According to a further or alternative aspect, the present invention provides a compound of formula (I) for use in the manufacture of a medicament for the treatment or prevention of a disease or condition in which pain and/or inflammation predominates.

The present invention also provides a method for the treatment or prevention of a disease or condition in which pain and/or inflammation predominates, which method comprises administration to a patient in need thereof of an effective amount of a compound of formula (I) or a composition comprising a compound of formula (I).

According to a further aspect of the present invention, it may be desirable to treat any of the aforementioned conditions with a combination of a compound according to the present invention and one or more other pharmacologically active agents suitable for the treatment of the specific condition. The compound of formula (I) and the other pharmacologically active agent(s) may be administered to a patient simultaneously, sequentially or in combination.

Thus, for example, for the treatment or prevention of pain and/or inflammation, a compound of the present invention may be used in conjunction with other analgesics, such as acetaminophen (paracetamol), aspirin and other NSAIDs, including selective cyclooxygenase-2 (COX-2) inhibitors, as well as opioid analgesics, especially morphine, NR2B antagonists, bradykinin antagonists, anti-migraine agents, anticonvulsants such as oxcarbazepine and carbamazepine, antidepressants (such as TCAs, SSRIs, SNRIs, substance P antagonists, etc.), spinal blocks, gabapentin, pregabalin and asthma treatments (such as Θ₂-adrenergic receptor agonists or leukotriene D₄antagonists (e.g. montelukast).

Specific anti-inflammatory agents include diclofenac, ibuprofen, indomethacin, nabumetone, ketoprofen, naproxen, piroxicam and sulindac, etodolac, meloxicam, rofecoxib, celecoxib, etoricoxib, parecoxib, valdecoxib and tilicoxib. Suitable opioid analgesics of use in conjunction with a compound of the present invention include morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine; or a pharmaceutically acceptable salt thereof. Suitable anti-migraine agents of use in conjunction with a compound of the present invention include CGRP-antagonists, ergotamines or 5-HT₁ agonists, especially sumatriptan, naratriptan, zolmatriptan or rizatriptan.

Therefore, in a further aspect of the present invention, there is provided a pharmaceutical composition comprising a compound of the present invention and an analgesic, together with at least one pharmaceutically acceptable carrier or excipient.

In a further or alternative aspect of the present invention, there is provided a product comprising a compound of the present invention and an analgesic as a combined preparation for simultaneous, separate or sequential use in the treatment or prevention of a disease or condition in which pain and/or inflammation predominates.

Compounds of formula I can be made by reacting a compound of formula I′ with a compound of formula XXX: 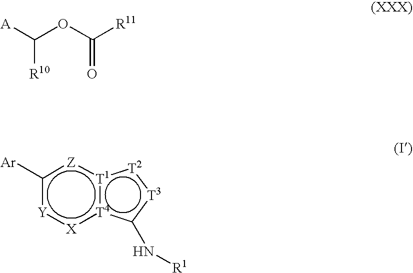
in which A, X, Y, Z, T¹, T², T³, T⁴, Ar, R¹, R¹⁰ and R¹¹ are as defined above, in an anhydrous solvent such as anhydrous acetonitrile, in the presence of a salt such as sodium iodide, for about 90° C. for about 15 h.

Compounds of formula I′ in which T³ and T⁴ are N can be made by reacting a compound of formula II with a compound of formula III: 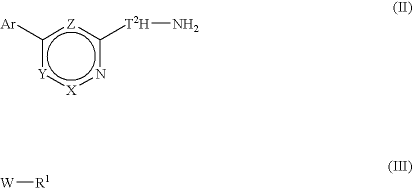
in which Ar, R¹, R², T², X, Y and Z are as defined above and W is an isocyanate or isothiocyanate group. When W is an isocyanate group the reaction is carried out in the presence of acetonitrile with heating to about 90° C. for about 12 h, followed by the addition of phosphorous oxychloride generally with heating at reflux for about 12 h, with this last step generally being repeated.

When W is an isothiocyanate group the reaction is generally heated to from 60 to 100° C. for about 1 h in a solvent such as p-xylene/N,N-dimethylacetamide after which an activating agent such as dicyclohexylcarbodiimide can be added with further heating at about 100° C. for about 1 h. The reaction can also be carried out in a solvent such as acetonitrile for about 15 h at about room temperature followed by heating with silver(I)acetate at about 150° C. for about 10 minutes in a microwave.

Compounds of formula II in which T² is N can be made by reacting a compound of formula IV: 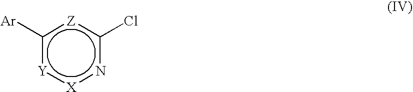
in which Ar, X, Y and Z are as defined above with hydrazine, usually as its monohydrate, in a solvent such as isopropanol at about 100° C. for about 15 h. This procedure can be repeated once or twice to improve yields.

Compounds of formula IV can be made by treating a compound of formula V with a compound of formula VI: 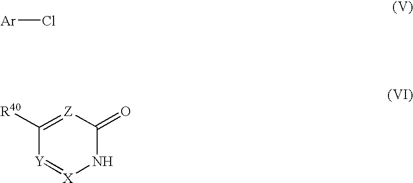
in which Ar, X, Y and Z are as defined above and R⁴⁰ is Cl or Sn(alkyl)₃, for example Sn(methyl)₃ or Sn(n-butyl)₃. When R⁴⁰ is Cl it can be initially converted into a group B(OH)₂ under conditions suitable for a Suzuki Coupling Reaction (for review, see for instance A. Suzuki, Pure Appl. Chem., 1991, 63, 419-422), for example, in the presence of a palladium catalyst such as tetrakis(triphenylphosphine)palladium (0), (1,1′-bis(diphenylphosphino)ferrocene)dichloropalladium or dichloro-(1,4-bis(diphenylphosphino)butane)palladium, in a suitable solvent such as an ether, for example, dimethoxyethane or dioxane or an aromatic hydrocarbon, for example toluene, at an elevated temperature and in the presence of a base such as sodium carbonate. Where R⁴⁰ is Sn(alkyl)₃, the reaction is conveniently effected under conditions suitable for a Stille Coupling Reaction (for review, see for instance J. K. Stille, Angew. Chem. Int. Ed., 1986, 25, 508-524), for example, in the presence of a palladium catalyst such as tetrakis(triphenylphosphine)palladium (0) or bis(triphenylphosphine)palladium (II) chloride, in a suitable solvent such as an ether, for example dioxane, or an aromatic hydrocarbon, for example, toluene, at an elevated temperature, and in the presence of catalysts such as LiCl and CuI.

The resulting compound can be converted to the desired chloride IV by reacting with phosphorous oxychloride at about 100° C. for about 1 h.

Alternatively compounds of formula IV can be made by reacting a compound of formula ArH with a compound of formula X: 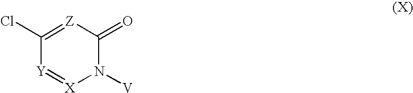
in which X, Y and Z are as defined above and V is a protecting group such as tetrahydropyranyl. The reaction is generally carried in the presence of a strong base such as BuLi, in the presence of zinc chloride and catalyst such as Pd(PPh₃)₄ in a solvent such as tetrahydrofuran between about −78° C. and room temperature for about 2h. The resulting product can be deprotected using phosphorous oxychloride with heating to about 90° C. for about 10 min.

Compounds of formula II in which T² is C(CH₂)ₙR² can be made by reacting a compound of formula VII: 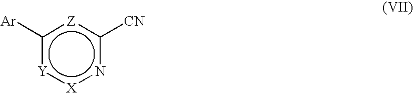
in which n, Ar, X, Y and Z are as defined above with ammonia in a hydrogenating environment, such as H₂/Pd/C, generally in a solvent such as methanol at about room temperature for about 1 h.

The nitrile of formula VII can be made by reacting the corresponding amide with a dehydrating agent such as Burgess reagent for up to 6 h in a solvent such as dichloromethane. This amide can be made from the corresponding carboxylic acid ester which is reacted with ammonia in a solvent such as methanol for about 3 h.

This carboxylic acid ethyl ester can be made from the corresponding compound of formula IV under an atmosphere of carbon monoxide in ethanol in the presence of a palladium catalyst such as Pd(dppf)Cl₂.CHCl₃ and a base such as sodium acetate at about 90° C. for about 2 h.

In an alternative route, compounds of formula I can be made by reacting a compound of formula VIII with a compound of formula IX: 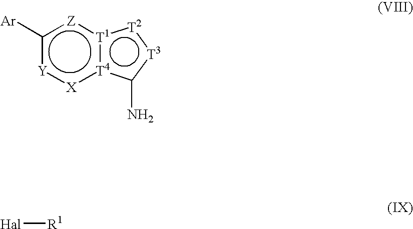
in which T¹, T², T³, T⁴, X, Y, Z, Ar and R¹ are as defined above and Hal is bromine or iodine. The reaction is generally carried out in the presence of a catalyst such as tris(dibenzylidene)dipalladium together with cesium carbonate in a solvent such as 1,4-dioxane at about 100° C. for from 15 min to 18 h. The reaction is promoted using a catalyst such as xantphos.

The compound of formula VIII can be made by reducing the corresponding nitro compound with, for example, Lindlar catalyst in MeOH:EtOAc on a Parr hydrogenator under H₂ for about 30 min.

This nitro compound can be made by nitrating a compound of formula XI: 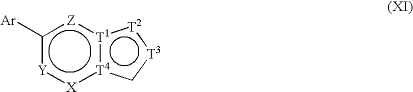
in which T¹, T², T³, T⁴, X, Y, Z and Ar are as defined above with, for example, a mixture of concentrated H₂SO₄ and fuming HNO₃ for about 30 min at about 0° C.

Compounds of formula XI in which T² and T⁴ are N and T³ is C(CH₂)ₙR² can be made by reacting a compound of formula XVII with bromoacetaldehyde or chloroacetaldehyde in a solvent such as ethanol in the presence of a mild base such as sodium hydrogencarbonate at about reflux for about 18 h. Bromoacetaldehyde can be made in situ by reacting bromoacetaldehydedimethylacetal with an acid such as hydrobromic acid in a solvent such as water.

The compound of formula XI can also be made by reacting a compound of formula V with a compound of formula XII: 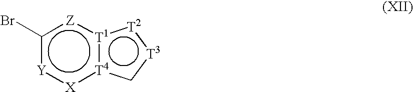
in which X, Y, Z, T¹, T², T³ and T⁴ are as defined above by a Suzuki reaction as described above, for example using bispinacolatodiborane.

Compounds of formula XII in which T¹=T²=X═N, T³=C(CH₂)ₙR² and Y=Z=C(CH₂)ₙR³ can be made by reacting a compound of formula XIII with a compound of formula XIV: 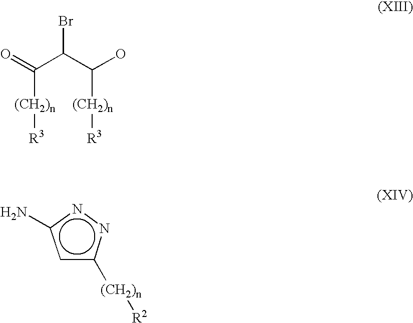
in which n, R² and R³ are as defined above, in the presence of acetic acid and in a solvent such as ethanol for about 4 h at reflux.

Compounds of formula XI can also be made by ring-closing a compound of formula II with, for example, formic acid at about 80° C. for about 30 min.

Compounds of formula VIII in which T²=T³=T⁴=N can be made by reacting a compound of formula IV with thiosemicarbazide generally in glacial acetic acid at about 135° C. for about 12 h.

An alternative route to producing compounds of formula IV in which X═N, Y═CCl and Z=CH is provided by reacting a compound of formula XV: 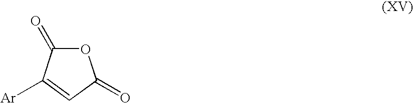
in which Ar is as defined above successively with hydrazine monohydrate and phosphorous oxychloride. The former reaction is generally carried out in glacial acetic acid with the gradual addition of concentrated sulphuric acid followed by heating to about 125° C. for about 3 h.

The compound of formula XV can be made by reacting a compound of formula XVI: 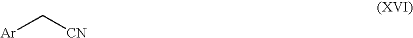
in which Ar is as defined above with glyoxylic acid monohydrate in a solvent such as methanol in the presence of a base such as potassium carbonate for about 15 h at about room temperature, followed by reacting with a mixture of formic acid and sulphuric acid generally at reflux for about 3 h.

Compounds of formula XI in which T²=T⁴=N and T³=CH can be made by reacting a compound of formula XVII: 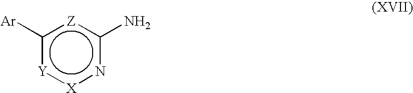
in which Ar, X, Y and Z are as defined above with chloroacetaldehyde generally in a solvent such as ethanol in the presence of a base such as sodium bicarbonate at reflux for about 18 h. Compounds of formula XVII can also be made by reacting a compound of formula XVIII with ammonia generally in a solvent such as water in a microwave at about 140° C. for about 30 minutes.

The compound of formula XVII can be made by reducing a compound of formula II in which T² is N for example with Raney Nickel under H₂ at about room temperature for about 48 h.

In an alternative method compounds of formula II can be made by reacting a compound of formula XVIII: 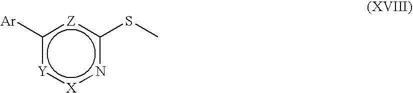
in which Ar, X, Y and Z are as defined above with hydrazine monohydrate in a solvent such as ethanol at reflux for about 16 h.

Compounds of formula XVIII in which X=Z=N and Y═CH can be made by reacting a compound of formula XIX: 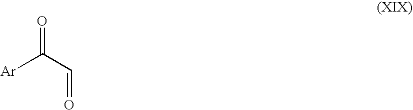
in which Ar is as defined above with aminomethanehydrazonathionate, generally as the hydroiodide salt, in a solvent such as water between about 0° C. and room temperature for about 1 h.

Compounds of formula I can also be made by reacting a compound of formula XX with a compound of formula XXI: 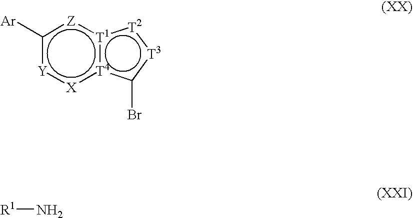
wherein T¹, T², T³, T⁴, X, Y, Z, Ar and R¹ are as defined above. The reaction is generally carried out in a solvent such as dioxane in the presence of an acid catalyst such as hydrobromic acid for about 15 min in a microwave.

The compound of formula XX can be made by brominating a compound of formula XI, for example using bromine in the presence of a buffered solution such as a mixture of acetic acid and sodium acetate at about 120° C. for about 2 h.

Compounds of formula I can be converted to other compounds of formula I by methods known in the art. Indeed, any of the intermediates can be functionalised by conventional methods. For example, compounds having an R³ group which is chlorine can be converted to compounds where that R³ group is hydrogen by reacting with ammonium formate in the presence of a catalyst such as Pd/C in a solvent such as anhydrous ethanol at about 80° C. for about 15 h.

Compounds in which Ar or Ar¹ is substituted by bromine can be converted into compounds where Ar or Ar¹ is substituted by an aromatic group by performing the appropriate Stille Coupling Reaction as described above.

Compounds having an acetyl group can be reacted with a methylating agent such as methyl magnesium bromide in a solvent such as tetrahydrofuran at a temperature of from −40° C. to 0° C. for about 15 h to produce the 2-hydroxyprop-2-yl analogue. Compounds in which the nitrogen atom of a pyridine moiety is oxidized can be made by reacting with, for example, oxone in a solvent such as chloroform in the presence of a catalyst such as aluminium oxide generally at reflux for about 18 h.

Compounds of formula I′ in which R² is H can be brominated to compounds of formula I′ in which R² is Br by reacting with a brominating agent such as N-bromosuccinimide in a solvent such as dichloromethane for about 5 min at about room temperature. This compound can undergo Suzuki Coupling Reactions to compounds of formula I in which R² is an aromatic group. The bromine atom can be replaced by a cyano group by reacting with zinc cyanide in the presence of a catalyst such as zinc powder and a coupling agent such as [1,1′-bis(diphenylphosphino)ferrocene]dichloropalladium(II)dichloromethane complex in a solvent such as N,N-dimethylacetamide at about 160° C. for about 20 min in a microwave. The cyano group can be converted to a formamide residue by hydrolyzing with, for example, concentrated hydrochloric acid at about 80° C. for about 20 min in a microwave. Compounds in which n in (CH₂)ₙR² is one and where R² is bound to the methylene group via a nitrogen atom, can be made by reacting a compound of formula I′ in which R² is hydrogen with formaldehyde and the nitrogen containing moiety, such as morpholine or dimethylamine, in the presence of an acid, such as acetic acid, in a solvent such as water at about room temperature for from 20 to 24 h. Compounds of formula I′ in which R² is carboxy can be made from compounds of formula I′ in which R² is bromine by reacting with carbon monoxide in ethanol in the presence of sodium acetate and a coupling agent such as [1,1′-bis(diphenylphosphino)ferrocene]dichloropalladium(II)dichloromethane complex at about reflux for about 3 h followed by hydrolysing the ester for example in a mixture of methanol, water and tetrahydrofuran in the presence of a base such as lithium hydroxide at about room temperature for about 24 h.

Intermediates for which no preparation is described above are commercially available or can be made from commercially available compounds by methods known in the art. The preparation of some of these intermediates is provided in the Descriptions and Examples.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P. G. M. Wuts, Protective Groups in Organic Synthesis, John Wiley & Sons, 1991. The protecting groups may be removed at a convenient subsequent stage using methods known from the art.

The following Examples serve to illustrate the preparation of compounds of the present invention.

### Common Intermediates

### Description 1

### 3-Chloro-5-(3-trifluoromethyl-2-pyridyl)pyridazine

To a mixture of 5-chloropyridazin-3-one (8.6 g, 62.9 mmol) and bis(pinacolato)diboron (16.8 g, 66.2 mmol) in anhydrous 1,4-dioxane (100 ml) was added bis(diphenylphosphino)ferrocenylpalladiumdichloride (2.3 g, 3.1 mmol) and potassium acetate (18.5 g, 188.5 mmol) and nitrogen was bubbled through the mixture for 10 min. The mixture was heated at 100° C. for 15 h, allowed to cool to room temperature and a mixture of 2-chloro-3-(trifluoromethyl)pyridine (10.9 g, 60 mmol) and bis(diphenylphosphino)ferrocenylpalladiumdichloride (2.3 g, 3.1 mmol) followed by 2M sodium carbonate (100 ml) was added to the black mixture and nitrogen was bubbled through for 10 min. The resulting mixture was heated at 100° C. for 15 h, allowed to cool to room temperature and poured into a mixture of ethyl acetate/ethanol/water (500/100/100 ml). The phases were separated and the aqueous phase was extracted two times with ethyl acetate (200 ml each). The combined organic layers were washed with brine, dried over sodium sulfate and adsorbed onto silica gel. Purification by flash chromatography (ethyl acetate) gave 5-(3-trifluoromethyl-2-pyridyl)pyridazin-3-one (4.9 g, 32%) as an off white solid, MS: (ES (M+1)) 242. The pyridazinone (4.8 g, 20 mmol) was suspended in phosphorous oxychloride (30 ml, 322 mmol) and the mixture was heated at 100° C. for 1 h. After cooling to room temperature the homogeneous dark solution was evaporated under reduced pressure and repartitioned between chloroform and water (50 ml each). The pH was adjusted to 8 by portionwise addition of saturated aqueous sodium carbonate solution and the phases were separated. After two further extractions the combined organic extracts were washed with water and brine and dried over sodium sulfate. After filtration the compound was adsorbed onto silica gel and purified by flash column (50% ethyl acetate-iso-hexane) to yield the title compound (3.9 g, 75%), MS: (ES (M+1)) 260/262.

¹H NMR (360 MHz, DMSO) δ 7.85 (1H, dd, J=7.5 and 4.5), 8.16 (1H, d, J=1.5), 8.45 (1H, d, J=7.5), 9.02 (1H, d, J=4.5), 9.43 (1H, d, J=1.5) ppm.

### Description 2

### N-(4-Trifluoromethylphenyl)-7-(3-trifluoromethyl-2-pyridyl)[1,2,4]triazolo[4,3-b]pyridazine-3-amine

To a mixture of Description 1 (3.5 g, 13.8 mmol) in anhydrous isopropanol (20 ml) was added hydrazine monohydrate (3,4 ml, 70 mmol) and the mixture was heated at 100° C. for 15 h. After cooling to room temperature the solution was concentrated under reduced pressure and toluene was added to the resulting oil. The mixture was concentrated under reduced pressure again and the whole procedure was repeated twice to yield 3-hydrazino-5-(3-trifluoromethyl-2-pyridyl)pyridazine (3.2 g, 91%) as a red syrup which crystallises over 3 days at room temperature.

The pyridazine (0.56 g, 2.2 mmol) was dissolved in dry acetonitrile (10 ml) and a solution of 4-trifluoromethylphenylisocyanate (0.43 g, 2.3 mmol) in 3 ml acetonitrile was added dropwise while stirring at room temperature. The solution was heated at 90° C. for 12 h and cooled to room temperature. Phosphorous oxychloride (0.41 ml, 4.4 mmol) was added dropwise to the suspension and the resulting mixture was heated under reflux for 12 h. After addition of more phosphorous oxychloride (0.41 ml, 4.4 mmol) the mixture was heated for another 12 h under reflux and tlc showed complete conversion of starting material. The resulting yellow solution was poured onto a mixture of chloroform and water (200/20 ml) and the pH was adjusted to 8 by portionwise addition of saturated aqueous sodium carbonate solution and the phases were separated. After two further extractions the combined organic extracts were washed with water and brine and dried over sodium sulfate. After filtration the compound was adsorbed onto silica gel and purified by flash column (50% ethyl acetate) to yield the title compound (0.45 g, 48%) as a canary-yellow solid, MS: (ES (M+1)) 425.

¹H NMR (360 MHz, DMSO) δ 7.70 (2H, d, J=8.7), 7.81 (1H, dd, J=8.0 and 4.6), 8.07 (2H, d, J=8.7), 8.37 (1H, d, J=1.4), 8.45 (1H, d, J=8.0), 8.77 (1H, d, J=1.4), 9.03 (1H, d, J=4.6), 10.32 (1H, s) ppm.

### Description 3

### 5-[3-Trifluoromethylpyridin-2-yl]pyridazin-3-amine

Raney Nickel (50% aq. suspension, 2 ml) was added to a solution of 3-hydrazino-5-[3-trifluoromethylpyridin-2-yl]pyridazine (from Description 2; 1.10 g, 4.31 mmol) in ethanol (100 ml). The mixture was then stirred under a balloon of hydrogen gas for 48 h. The catalyst was then filtered off on a glass fibre pad, washing the solid thoroughly with ethanol. The filtrate was evaporated and the residue was then purified using a strong cation exchange (SCX) ion exchange cartridge washing away non-basic impurities with methanol, then eluting with 2M methanolic ammonia solution. Evaporation of the basic fraction gave the title compound as a red-brown solid (573 mg). ¹H NMR (400 MHz, DMSO) δ 8.97 (1H, br. d, J 5), 8.48 (1H, d, J 2), 8.37 (1H, d, J 8), 7.75 (1H, dd, J 8, 5), 6.82 (1H, d, J 2), 6.60 (2H, br. s); m/z (ES⁺) 241 (M+H⁺).

### Description 4

### 7-[3-Trifluoromethylpyridin-2-yl]imidazo[1,2-b]pyridazine

Description 3 (570 mg, 2.38 mmol) was dissolved in ethanol (10 ml). Sodium bicarbonate (400 mg, 4.75 mmol) was then added followed by chloroacetaldehyde (45% aq. solution, 450 μl, ca. 3.25 mmol) and the reaction mixture was heated at reflux for 18 h. After cooling to room temperature, flash silica was added, the solvent removed and the residue purified by flash column chromatography (eluant 1:19 MeOH—CH₂Cl₂) to give the title compound. ¹H NMR (400 MHz, DMSO) δ 9.01 (1H, d, J 5), 8.68 (1H, d, J 2), 8.44 (1H, br. s), 8.42 (1H, d, J 8), 8.25 (1H, br. s), 7.93 (1H, s) 7.78 (1H, dd, J 8, 5); m/z (ES⁺) 265 (M+H⁺).

### Description 5

### 3-Nitro-7-[3-trifluoromethylpyridin-2-yl]imidazo1,2-b]pyridazine

Description 4 (337 mg, 1.28 mmol) was dissolved in conc. sulfuric acid (3 ml) at 0° C. A nitrating mixture of conc. sulfuric acid and fuming nitric acid (1:1, 2 ml) was then added dropwise over 10 min. The mixture was then allowed to warm to room temperature and stir for 20 h before pouring into ice-water (150 ml). The mixture was made basic by addition of 33% aqueous ammonia solution, then extracted with ethyl acetate (3×30 ml). The combined organic layers were dried (Na₂SO₄) and evaporated and the residue purified by flash column chromatography (eluant 1:19 MeOH—CH₂Cl₂) to give the title compound (240 mg) as a colourless solid. ¹H NMR (400 MHz, DMSO) δ 9.14 (1H, d, J 2), 9.06 (1H, d, J 5), 8.93 (1H, s), 8.2 (1H, d, J 2), 8.47 (1H, d, J 8), 7.84 (1H, dd, J 8, 5); m/z (ES⁺) 310 (M+H⁺).

### Description 6

### 7-[3-Trifluoromethylpyridin-2-yl]imidazo[1,2-b]pyridazin-3-amine

Lindlar catalyst (100 mg) slurried in ethanol (1 ml) was added to a solution of Description 5 (170 mg, 0.55 mmol) in an ethanol-ethyl acetate mixture (1:1, 10 ml). The reaction mixture was then stirred under a balloon of hydrogen gas at room temperature for 5 h. The mixture was then filtered, washing the catalyst with ethanol (5 ml) and the filtrate was then evaporated. Addition of toluene (5 ml) to the residue and re-evaporation gave the title compound (153 mg) as a red oil which was free of ethanol and used without further purification. ¹H NMR (500 MHz, DMSO) 88.98 (1H, d, J 5), 8.54 (1H, s), 8.38 (1H, d, J 8), 7.96 (1H, s), 7.71 (1H, dd, J 8, 5), 7.21 (1H, s), 5.74 (2H, s); m/z (ES⁺) 280 (M+H⁺).

### Description 7

### N-[4-Trifluoromethylphenyl]-7-[3-trifluoromethylpyridin-2-yl]imidazo[1,2-b]pyridazin-3-amine

A mixture of Description 6 (150 mg, 0.55 mmol), 4-bromobenzotrifluoride (125 mg, 77 μl, 0.55 mmol) and caesium carbonate (254 mg, 0.78 mmol) in 1,4-dioxan (5 ml) was degassed (N₂×3), then 9,9-dimethyl-4,5-bis(diphenylphosphino)xanthene [xantphos] (19.3 mg, 0.033 mmol) and tris(dibenzylideneacetone)dipalladium (0) (10.2 mg, 0.011 mmol) were added and the mixture degassed again (N₂×3). The reaction was then heated to 100° C. for 24 h under nitrogen, then cooled to room temperature and diluted with tetrahydrofuran (20 ml). The mixture was then filtered through a glass fibre pad and the filtrate evaporated. The residue was purified by flash column chromatography (eluant 1:39 MeOH—CH₂Cl₂) and also by mass-directed preparative hplc to give the title compound as a yellow-orange solid (115 mg). ¹H NMR (500 MHz, DMSO) δ 9.06 (1H, s), 9.02 (1H, d, J 5 Hz), 8.70 (1H, d, J 1.5 Hz), 8.43 (1H, d, J 8 Hz), 8.24 (1H, d, J 1.5 Hz), 7.94 (1H, s), 7.78 (1H, dd, J 8, 5 Hz), 7.53 (2H, d, J 8 Hz), 6.96 (2H, d, J 8 Hz); MS: (ES (M+1)), 424.

### Description 8

### 5-(3-Trifluoromethylpyridin-2-yl)pyridazine-3-carboxylic acid ethyl ester

To a solution of Description 1 (0.50 g, 1.93 mmol) in ethanol in a 3-neck flask equipped with a condenser and a bubbler was added sodium acetate (0.32 g, 3.86 mmol). Nitrogen was bubbled through the resulting solution for 10 min. Pd(dppf)Cl₂.CHCl₃ (0.10 g, 0.14 mmol) was added and the reaction flushed with carbon monoxide. After 5 min of rapid CO bubbling the orange solution had darkened. The gas flow rate was reduced and the reaction heated to 90° C. After 2 h the starting material had been consumed and the solution was flushed with nitrogen. The reaction was condensed, partitioned between pH 7 phosphate buffer and ethyl acetate and the aqueous layer washed again with ethyl acetate. The organic layers were combined, dried over MgSO₄ and the crude product purified by flash column chromatography, eluting with 50% to 25% hexane in ethyl acetate to give the ethyl ester (0.37 g, 66%). m/z (ES⁺) 297 (M+H⁺). ¹H NMR (400 MHz, CDCl₃) 1.51 (3H, t, J 7.1), 4.59 (2H, q, J 7.1), 7.63 (1H, m), 8.20 (1H, dd, J 8.1, 0.8), 8.38 (1H, d, J 2.1), 8.96 (1H, d, J 0.7), 9.51 (1H, d, J 2.1).

### Description 9

### 5-(3-Trifluoromethylpyridin-2-yl)pyridazine-3-carboxylic acid amide

To Description 8 (150 mg) was added a solution of ammonia in methanol (2 M, 10 ml) and the reaction stirred for 3 h. The reaction was condensed to yield the desired amide (140 mg, 100%). m/z (ES⁺) 269 (M+H⁺). ¹H NMR (400 MHz, CDCl₃) 5.96 (1H, s), 7.61 (1H, ddd, J 7.8, 4.7, 0.9), 8.07 (1H, s), 8.19 (1H, dd, J 7.9, 1.0), 8.50 (1H, d, J 2.2), 8.96 (1H, d, J 5.0), 9.47 (1H, d, J 2.2).

### Description 10

### 4-Trifluoromethylphenyl-3-(3-trifluoromethylpyridin-2-yl)imidazo[1,5-b]pyridazin-7-ylamine

To a solution of Description 9 (5 mg) in dichloromethane (0.5 ml) was added Burgess reagent (9 mg) in 3 portions over 1 h. After 3 h an additional 3 mg Burgess reagent added. After 6 h the reaction was condensed and the product isolated by gradient column chromatography, eluting with 50% ethyl acetate in hexane-neat ethyl acetate to give the desired nitrile (4 mg). ¹H NMR (400 MHz, CDCl₃) 7.59 (1H, ddd, J 8.2, 4.1, 1.3 Hz), 7.95 (1H,d, J 2.2 Hz), 8.15 (1H, dd, J 8.0, 1.0 Hz), 8.91 (1H, s), 9.48 (1H, d, J=2.2 Hz). The nitrile (4 mg) was taken up in a solution of ammonia in methanol (2 M, 0.75 ml). 2 drops of a slurry of 10% Pd/C in water were added and the reaction stirred under a balloon of hydrogen. After 1 h the product had been consumed and the reaction was filtered and the filtrate condensed in vacuo. The crude amine was taken up in toluene (1 ml) and 4-trifluoromethyl phenylisothiocyanate (4 mg) was added and the reaction stirred at room temperature for 2 h. A further isothiocyanate (1 mg) was added and the reaction stirred an additional 90 min. Dicyclohexylcarbodiimide (4 mg) was added and the reaction heated to 100° C. After 45 min the reaction was condensed and purified by gradient column chromatography, eluting with 3:1 to 1:1 hexane:ethyl acetate followed SCX column chromatography eluting with methanol then ammonia in methanol (2M) to give the desired imidazopyridizine (2.75 mg). MS: (ES (M+1)), 424 ¹H NMR (500 MHz, MeOH-d₄) 7.46 (1H, s), 7.57 (2H, d, J 8.6 Hz), 7.65 (1H, ddd, J 8.1, 4.9, 0.8 Hz), 7.76 (2H, d, J 8.5 Hz), 8.04 (1H, d, J 2.1 Hz), 8.27 (1H, d, J 2.2 Hz), 8.30 (1H, dd, J 8.1, 1.3 Hz), 8.90 (1H, s).

### Final Products

### EXAMPLE 1

### 5-(2,2-Dimethylpropanoyloxymethyl)-3-(4-trifluoromethylphenylamino)-7-(3-trifluoromethyl-2-pyridyl)-1,2,4]triazolo[4,3-b]pyridazinium chloride

To a mixture of Description 2 (0.712 g, 1.68 mmol) in anhydrous acetonitrile (10 ml) was added chloromethyl pivalate (1.22 ml, 8.40 mmol) and sodium iodide (25 mg, 0.17 mmol) and the mixture was heated at 90° C. for 15 hours. After cooling to room temperature the solution was concentrated under reduced pressure and diethyl ether (10 ml) was added to the resulting brown gum to induce crystallisation. The resulting solid was filtered and washed with additional diethyl ether and dried on the sinter to yield a brown solid which was dissolved in dimethylsulfoxide and submitted to reverse phase HPLC purification (using aqueous diethylamine/acetonitrile gradients). The purple product fractions were combined and evaporated to dryness under reduced pressure to yield 375 mg of a purple solid which was dissolved in acetonitrile (5 ml) and a solution of 1 M hydrogen chloride in diethyl ether was added until the colour changed from purple to bright yellow (0.7 ml; 0.7 mmol). Diethyl ether was added (5 ml) to induce crystallization and the yellow product was filtered and washed with diethyl ether (5 ml) and dried on the sinter to yield the target compound (0.36 g; 37%) as a bright yellow solid, MS: (ES (M+)) 539. ¹H NMR (360 MHz, DMSO) δ1.17 (9H, s), 6.60 (2H, s), 7.85 (2H, d, J=8.7), 7.95 (1H, dd, J=8.0 and 4.6), 8.01 (2H, d, J=8.7), 8.58 (1H, d, J=8.0), 9.14 (1H, d, J=4.6), 9.17 (1H, d, J=1.6), 9.49 (1H, d, J=1.6), 11.46 (1H, s) ppm.

### EXAMPLE 2

### 5-(1-(2,2-Dimethylpropanoyloxy)-1-ethyl)-3-(4-trifluoromethylphenylamino)-7-(3-trifluoromethyl-2-pyridyl)-[1,2,4]triazolo[4,3-b]pyridazinium chloride

A procedure analogous to that of Example 1 gave the target compound (0.045 g, 25%) as a bright yellow solid, MS: (ES (M+)) 553. ¹H NMR (500 MHz, DMSO) δ 1.15 (9H, s), 1.92 (3H, d, J=6.0), 7.47 (1H, q, J=6.0), 7.85 (2H, d, J=8.0), 7.97 (1H, dd, J=8.0 and 4.6), 8.01 (2H, d, J=8.6), 8.57 (1H, d, J=8.0), 9.13 (2H, m), 9.45 (1H, d, J=1.3), 11.42 (1H, s) ppm.

### EXAMPLE 3

### 5-(1-Acetoxy-1-ethyl)-3-(4-trifluoromethylphenylamino)-7-(3-trifluoromethyl-2-pyridyl)-[1,2,4]triazolo[4,3-b]pyridazinium chloride

A procedure analogous to that of Example 1 gave the target compound (0.038 g, 20%) as a bright yellow solid, MS: (ES (M+)) 511. ¹H NMR (500 MHz, DMSO) δ 1.92 (3H, d, J=6.0), 2.07 (3H, s), 7.52 (1H, q, J=6.0), 7.85 (2H, d, J=7.9), 7.95 (1H, m), 8.03 (2H, d, J=7.9), 8.57 (1H, d, J=8.3), 9.13 (2H, m), 9.45 (1H, s), 11.43 (1H, s) ppm.

### EXAMPLE 4

### 5-(2-Methylpropanoyloxymethyl)-3-(4-trifluoromethylphenylamino)-7-(3-trifluoromethyl-2-pyridyl)-[1,2,4]triazolo[4,3-b]pyridazinium chloride

A procedure analogous to that of Example 1 gave the target compound (0.065 g, 37%) as a bright yellow solid, MS: (ES (M+)) 525. ¹H NMR (500 MHz, DMSO) δ 1.11 (6H, d, J=7.0), 2.64 (1H, q, J=7.0), 6.61 (2H, s), 7.85 (2H, d, J=8.7), 7.96 (1H, dd, J=8.0 and 5.0), 8.02 (2H, d, J=8.7), 8.57 (1H, d, J=8.0), 9.14 (1H, d, J=5.0), 9.18 (1H, d, J=1.5), 9.48 (1H, d, J=1.5), 11.48 (1H, s) ppm.

### EXAMPLE 5

### 5-(1-Pyrrolidinylcarbonyloxymethyl)-3-(4-trifluoromethylphenylamino)-7-(3-trifluoromethyl-2-pyridyl)-[1,2,4]triazolo[4,3-b]pyridazinium chloride

A procedure analogous to that of Example 1 gave the target compound (0.13 g, 42%) as a bright yellow solid, MS: (ES (M+)) 552. ¹H NMR (500 MHz, DMSO) δ 1.79 (4H, m), 3.24 (2H, t, J=6.6), 3.35 (2H, t, J=6.6), 6.55 (2H, s), 7.85 (2H, d, J=8.7), 7.95 (1H, dd, J=8.0 and 5.1), 8.03 (2H, d, J=8.7), 8.57 (1H, d, J=8.0), 9.12 (1H, d, J=1.5), 9.15 (1H, d, J=5.1), 9.45 (1H, d, J=1.5), 11.42 (1H, s) ppm.

### EXAMPLE 6

### 5-(1-(2-Methylpropanoyloxy)-1-ethyl)-3-(4-trifluoromethylphenylamino)-7-(3-trifluoromethyl-2-pyridyl)-[1,2,4]triazolo[4,3-b]pyridazinium chloride

A procedure analogous to that of Example 1 gave the target compound (0.135 g, 48%) as a bright yellow solid, MS: (ES (M+)) 539. ¹H NMR (500 MHz, DMSO) δ 1.05 (3H, d, J=7.0), 1.12 (3H, d, J=7.0), 1.92 (3H, d, J=6.0), 2.60 (1H, sept, J=7.0), 7.50 (1H, q, J=6.0), 7.84 (2H, d, J=8.7), 7.96 (1H, dd, J=8.0 and 5.0), 8.03 (2H, d, J=8.7), 8.57 (1H, d, J=8.0), 9.14 (2H, m), 9.45 (1H, d, J=1.5), 11.44 (1H, s) ppm.

### EXAMPLE 7

### 5-(1-(1-Methyl-1-ethoxycarbonyloxy)-1-ethyl)-3-(4-trifluoromethylphenylamino)-7-(3-trifluoromethyl-2-pyridyl)-[1,2,4]triazolo[4,3-b]pyridazinium chloride

A procedure analogous to that of Example 1 gave the target compound (0.14 g, 39%) as a bright yellow solid, MS: (ES (M+)) 555. ¹H NMR (500 MHz, DMSO) δ 1.17 (3H, d, J=6.3), 1.24 (3H, d, J=6.3), 1.94 (3H, d, J=6.0), 4.74 (1H, sept, J=6.3), 7.46 (1H, q, J=6.0), 7.85 (2H, d, J=8.7), 7.96 (1H, dd, J=8.1 and 5.0), 8.05 (2H, d, J=8.7), 8.57 (1H, d, J=8.1), 9.13 (1H, d, J=1.5), 9.16 (1H, d, J=5.0), 9.46 (1H, d, J=1.5), 11.49 (1H, s) ppm.

### EXAMPLE 8

### 1-{[2,2-Dimethylpropanoyloxy]methyl}-3-{4-trifluoromethylphenylamino}-7-[3-trifluoromethylpyridin-2-yl]imidazo[1,2-b]pyridazin-1-ium chloride

A procedure analogous to that of Example 1 using Description 7 gave the target compound (0.07 g, 23%) as a bright yellow solid, MS: (ES (M+)) 538. ¹H NMR (500 MHz, DMSO) δ 1.17 (9H, s), 6.50 (2H, s), 7.22 (2H, d, J=8.5), 7.63 (2H, d, J=8.7), 7.92 (1H, dd, J=4.9, 8.1), 8.54 (1H, d, J=8.1), 8.76 (1H, s), 9.10 (1H, d, J=4.5), 9.17 (1H, d, J=1.8), 9.37 (1H, d, J=1.7), 9.74 (1H, s).

### EXAMPLE 9

### 6-{[2,2-Dimethylpropanoyloxy]methyl}-7-{4-trifluoromethylphenylamino}-3-[3-trifluoromethylpyridin-2-yl]imidazo[1,5-b]pyridazin-6-ium chloride

A procedure analogous to that of Example 1 using Description 10 gave the target compound (0.06 g, 20%) as a yellow solid, MS: (ES (M+)) 538.

### Biological Methodology

Determination of In Vitro Activity

CHO cells, stably expressing recombinant human VR1 receptors and plated into black-sided 384-well plates, were washed twice with assay buffer (Hepes-buffered saline) and then incubated with 1 uM Fluo-3-AM for 60 minutes in darkness. Cells were washed twice more to remove excess dye, before being placed, along with plates containing capsaicin and test compounds in a Molecular Devices FLIPR. The FLIPR simultaneously performed automated pharmacological additions and recorded fluorescence emmission from Fluo-3. In all experiments, basal fluorescence was recorded, before addition of test compounds and subsequent addition of a previously determined concentration of capsaicin that evoked 80% of the maximum response. Inhibition of capsaicin evoked increases in intracellular [Ca²⁺] were expressed relative to wells on the same plate to which capsaicin was added in the absence of test compounds. Increases in intracellular [Ca²⁺] occurring after addition of test compound alone, prior to addition of capsaicin, allow determination of intrinsic agonist or partial agonist activity, if present. All the above compounds had an IC₅₀ of below 2 μM.

Determination of In Vivo Efficacy in a Capsaicin Paw Flinch Model

(Method adapted from Taniguchi et al, 1997, Br J Pharmacol. 122(5):809-12) T0 determine in vivo functional occupancy of VR1 receptors, compounds are administered orally to male Sprague Dawley rats typically 1 hour prior to receiving an intraplantar injection of capsaicin (2 Tg dissolved in ethanol) and the number of flinches of the injected paw is recorded for 5 minutes immediately thereafter. Statistical analysis is performed using one-way ANOVA followed by Dunnett's test; p values <0.05 compared to capsaicin/vehicle-treated rats are considered significant.

Determination of In Vivo Efficacy in a Model of Inflammatory Pain

(Method adapted from Hargreaves et al, 1988 Pain, 32(1):77-88). Antinociceptive activity is determined using a rat carrageenan-induced thermal hyperalgesia assay. Inflammatory hyperalgesia is induced by intraplantar injection of carrageenan (lambda-carrageenan 0.1 ml of 1% solution made up in saline) into one hind paw. Compounds are given orally typically 2 hours after carrageenan and paw withdrawal latencies determined 1 hour later. Paw withdrawal latencies to application of noxious thermal stimuli to plantar surface of the hind paw are measured using the Hargreaves apparatus. Thermal hyperalgesia is defined as the difference in paw withdrawal latencies for saline/vehicle- and carrageenan/vehicle-treated rats. Paw withdrawal latencies for drug treated rats are expressed as a percentage of this response. Statistical analysis is performed using one-way ANOVA followed by Dunnett's test; p values <0.05 compared to carrageenan/vehicle-treated rats are considered significant.

1. A compound of formula (I): 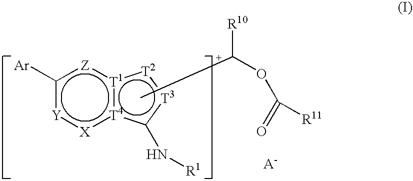
wherein:
one of T¹ and T⁴ is N and the other is C;
one of T² and T³ is N and the other is C(CH₂)ₙR² or N;
X, Y and Z are independently N or C(CH₂)ₙR³;
R¹ is Ar¹ or R¹ is C₁₋₆alkyl optionally substituted with one or two groups Ar¹;
Ar¹ is cyclohexyl, piperidinyl, piperazinyl, morpholinyl, adamantyl, phenyl, naphthyl, a six-membered heteroaromatic ring containing one, two or three nitrogen atoms, a five-membered heteroaromatic ring containing one, two, three or four heteroatoms chosen from O, N and S, at most one O or S atom being present, or a nine- or ten-membered bicyclic heteroaromatic ring in which phenyl or a six-membered heteroaromatic ring as defined above is fused to a six- or five-membered heteroaromatic ring as defined above;
Ar¹ is optionally substituted by one, two or three groups chosen from halogen, hydroxy, cyano, nitro, isonitrile, CF₃, OCF₃, SF₅, C₁₋₆alkyl, C₂₋₆alkenyl, C₂₋₆alkynyl, C₁₋₆alkoxy, C₁₋₆alkylthio, C₁₋₆alkylsulfinyl, C₁₋₆alkylsulfonyl, —NR⁶R⁷, CONR⁶R⁷, —COH, —CO₂H, C₁₋₆alkylcarbonyl, C₁₋₆alkoxycarbonyl, haloC₁₋₆alkyl, haloC₂₋₆alkenyl, haloC₁₋₆alkoxy, hydroxyC₁₋₆ alkyl, aminoC₁₋₆alkyl, cyanoC₁₋₆alkyl, C₃₋₆cycloalkyl, hydroxyC₃₋₆cycloalkyl, aminoC₃₋₆cycloalkyl, haloC₃₋₆cycloalkyl, cyanoC₃₋₆cycloalkyl, haloC₁₋₆alkylcarbonyl, C₁₋₆alkoxycarbonylC₁₋₆alkyl, (halo)(hydroxy)C₁₋₆alkyl, (halo)(hydroxy)C₃₋₆cycloalkyl, phenyl and a five-membered heteroaromatic ring containing one, two or three heteroatoms, at most one O or S atom being present; wherein the phenyl and five-membered heteroaromatic ring are optionally substituted by C₁₋₆alkyl, halo, hydroxy or cyano; when two C₁₋₆alkyl groups substitute adjacent positions on Ar¹ then, together with the carbon atoms to which they are attached, they may form a partially saturated ring containing five or six carbon atoms; when two C₁₋₆alkoxy groups substitute adjacent positions on Ar¹ then, together with the carbon atoms to which they are attached, they may form a partially saturated five- or six-membered ring;
Ar is phenyl, a six-membered heteroaromatic ring containing one, two or three nitrogen atoms or a five-membered heteroaromatic ring containing one, two, three or four heteroatoms chosen from O, N and S, at most one heteroatom being O or S, Ar being optionally substituted by one, two or three groups chosen from halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, haloC₁₋₆alkoxy, hydroxyC₁₋₆alkyl, aminoC₁₋₆alkyl, C₁₋₆alkylcarbonyl and a five-membered heteroaromatic ring containing one, two, three or four heteroatoms chosen from O, N and S, at most one heteroatom being O or S, optionally substituted by C₁₋₆alkyl, halogen, amino, hydroxy or cyano;
R² and R³ are independently hydrogen, halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl, aminoC₁₋₆alkyl, C₁₋₆alkylaminoC₁₋₆alkyl, di(C₁₋₆alkyl)aminoC₁₋₆alkyl, amido, piperidinyl, piperazinyl, C₃₋₆cycloalkyl, morpholinyl, phenyl, a six-membered heteroaromatic ring containing one, two or three nitrogen atoms or a five-membered heteroaromatic ring containing one, two, three or four heteroatoms chosen from O, N and S, at most one O or S atom being present, which phenyl, six-membered heteroaromatic ring and five-membered heteroaromatic ring are optionally substituted by haloC₁₋₆alkyl, C₁₋₆alkyl, hydroxy, halogen, amino or cyano;
R⁶ and R⁷ are independently hydrogen or C₁₋₆alkyl; when both R⁶ and R⁷ are C₁₋₆alkyl then, together with the nitrogen atom to which they are attached, they may form a five or six membered saturated nitrogen containing ring;
n is zero, one, two or three;
R¹⁰ and R¹¹ are independently hydrogen, C₁₋₆alkyl, C₁₋₆alkoxy, C₃₋₆cycloalkyl or NR¹²R¹³;
R¹² and R₁₃ are independently hydrogen or C₁₋₆alkyl or R¹² and R¹³, together with the nitrogen atom to which they are attached, may form a nitrogen containing heterocycle; and
A⁻ is a pharmaceutically acceptable anion.

2. A compound according to claim 1 of formula (I″): 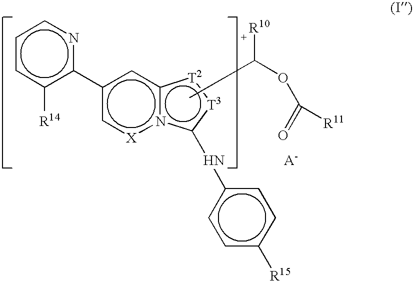
wherein:
X is CH or N;
one of T² and T³ is N and the other is C(CH₂)ₙR²;
R² is hydrogen, halogen, CF₃, OCF₃, C₁₋₆alkyl, C₂₋₆alkenyl, C₂₋₆alkynyl, nitro, cyano, isonitrile, hydroxy, C₁₋₆alkoxy, C₁₋₆alkylthio, —NR⁶R⁷, —CONR⁶R⁷, —COH, CO₂H, C₁₋₆alkoxycarbonyl, haloC₁₋₆alkyl, hydroxyC₁₋₆alkyl, aminoC₁₋₆alkyl, C₁₋₆alkylaminoC₁₋₆alkyl, di(C₁₋₆alkyl)aminoC₁₋₆alkyl, amido, piperidinyl, piperazinyl, C₃₋₆cycloalkyl, morpholinyl, phenyl, a six-membered heteroaromatic ring containing one, two or three nitrogen atoms or a five-membered heteroaromatic ring containing one, two, three or four heteroatoms chosen from O, N and S, at most one O or S atom being present, which phenyl, six-membered heteroaromatic ring and five-membered heteroaromatic ring are optionally substituted by haloC₁₋₆alkyl, C₁₋₆alkyl, hydroxy, halogen, amino or cyano;
R⁶ and R⁷ are independently hydrogen or C₁₋₆alkyl; when both R⁶ and R⁷ are C₁₋₆alkyl then, together with the nitrogen atom to which they are attached, they may form a five or six membered saturated nitrogen containing ring;
n is zero, one, two or three;
R¹⁰ and R¹¹ are independently hydrogen, C₁₋₆alkyl, C₁₋₆alkoxy, C₃₋₆cycloalkyl or NR¹²R¹³ where R¹² and R¹³ are independently hydrogen or C₁₋₆alkyl or R¹² and R¹³, together with the nitrogen atom to which they are attached, form a nitrogen-containing heterocycle;
R¹⁴ and R¹⁵ are independently C₁₋₆alkyl, CF₃, haloC₁₋₆alkyl, halogen, C₁₋₆alkoxy, haloC₁₋₆alkoxy or OCF₃; and
A⁻ is a pharmaceutically acceptable anion.

3. A compound according to claim 1 of formula (IA): 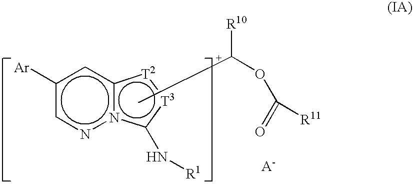
wherein T², T³, Ar, R¹, R¹⁰, R¹¹ and A⁻ are as defined in claim 1.

4. A compound according to claim 1 of formula (IB): 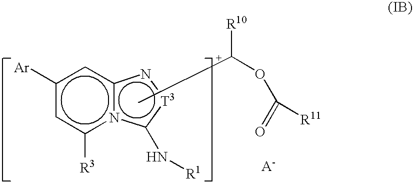
wherein Ar, R¹, R³, T³, R¹⁰, R¹¹ and A⁻ are as defined in claim 1.

5. A compound according to claim 1 of formula (IC): 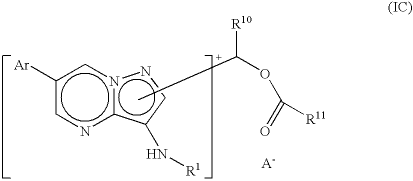
wherein Ar, R¹, R¹⁰, R¹¹ and A⁻ are as defined in claim 1.

6. A compound according to claim 1 of formula (ID): 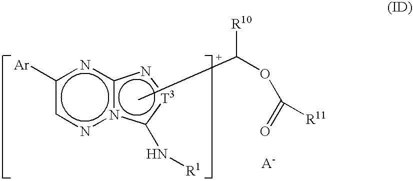
wherein Ar, R¹, T³, R¹⁰, R¹¹ and A⁻ are as defined in claim 1.

7. A compound according to claim 1 of formula (IE): 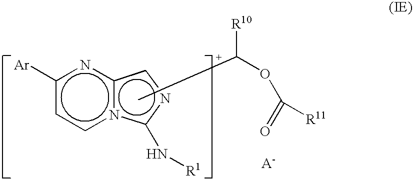
wherein Ar, R¹, R¹⁰, R¹¹ and A⁻ are as defined in claim 1.

8. A pharmaceutical composition comprising one or more compounds according to any one of claims 1 to 7 in association with a pharmaceutically acceptable carrier or excipient.

9. A compound according to any one of claims 1 to 7 for use in treatment of the human or animal body.

10. A compound according to any one of claims 1 to 7 for use in the manufacture of a medicament for the treatment or prevention of physiological disorders that may be ameliorated by modulating VR1 activity.

11. A compound according to any one of claims 1 to 7 for use in the manufacture of a medicament for the treatment or prevention of a disease or condition in which pain and/or inflammation predominates.

12. A method for the treatment or prevention of physiological disorders that may be ameliorated by modulating VR1 activity, which method comprises administration to a patient in need thereof of an effective amount of a compound of claim 1 or a composition comprising a compound of claim 1.

13. A method for the treatment or prevention of a disease or condition in which pain and/or inflammation predominates, which method comprises administration to a patient in need thereof of an effective amount of a compound of claim 1 or a composition comprising a compound of claim 1.
